(19) United States
(12) Patent Application Publication
(10) Pub. No.: US 2008/0025986 A1
(43) Pub. Date: Jan. 31, 2008

(54) Methods of Treating Tnf-Mediated Disorders

(76) Inventors: Osman N. Ozes, San Bruno, CA (US); Lawrence M. Blatt, San Francisco, CA (US)

Correspondence Address: KNOBBE MARTENS OLSON & BEAR LLP, 2040 MAIN STREET, FOURTEENTH FLOOR, IRVINE, CA, 92614, US

(21) Appl. No.: 10/557,647

(22) PCT Filed: Jun. 3, 2004
(86) PCT No.: PCT/US04/17728

§ 371 Date: Dec. 18, 2006

Related U.S. Application Data
(60) Provisional application No. 60/476,711, filed on Jun. 6, 2003.

(51) Int. Cl. A61K 39/395; A61K 31/4412; A61K 38/00; A61P 3/10; A61P 29/00; A61K 38/28

(52) U.S. Cl. 424/145.1; 514/2; 514/345; 514/4

(57) ABSTRACT

The present invention provides methods of treating TNF-α-mediated disorders, the methods generally involving administering to an individual in need thereof effective amounts of pirfenidone or a pirfenidone analog and a second therapeutic agent that reduces TNF-α synthesis or that reduces TNF-α binding to a TNF receptor. The present invention further provides methods for treating non-alcoholic steatohepatitis, the method generally involving administering to an individual in need thereof an effective amount of pirfenidone. The present invention further provides methods of treating end-stage or advanced Type II diabetes, the methods generally involving administering to an individual in need thereof effective amounts of pirfenidone and insulin.

### CROSS-REFERENCE

This application claims the benefit of U.S. Provisional Patent Application No. 60/476,711 filed Jun. 6, 2003, which application is incorporated herein by reference in its entirety.

### FIELD OF THE INVENTION

The present invention is in the field of treatments for TNF-mediated disorders.

### BACKGROUND OF THE INVENTION

The pathology of a variety of disorders is attributed to excessive amounts of TNF-α, either locally or systemically. For example, there is strong evidence that abnormally high production and release from cells of TNF-α contributes to disease initiation and progression in rheumatoid arthritis, systemic inflammatory syndromes, diabetes, and multiple sclerosis. In every one of these conditions, the initiating and sustaining pathophysiologic action is directly a result of an immediate local release and synthesis of massive amounts of TNF-α from several types of cells at or adjacent to the site of tissue damage. The locally released TNF-α is followed by additional synthesis and release of TNF-α by invading macrophages drawn to the site of tissue damage by a cascade of chemotactic cytokines released locally from cells in response to the greatly elevated TNF-α concentrations.

Obesity is a risk factor Type II diabetes and for non-alcoholic steatohepatitis (NASH). Insulin resistance is a common feature of obesity and predisposes the affected individuals to a variety of diseases, including hypertension, dyslipidemias, cardiovascular problems and Type II diabetes mellitus. It has been postulated that TNF-α-mediated serine-threonine phosphorylation of Insulin Receptor Substrates (ISR) contributes significantly to TNF-α-induced diabetes. Insulin resistance represents the most reproducible predisposing factor for NASH.

NASH is increasingly recognized as a relatively prevalent disorder that can lead to cirrhosis in some individuals. In fact, nearly 20% of patients with histologically proven NASH progress to cirrhosis. NASH can also progress to hepatic insufficiency and hepatocellular carcinoma. Because this disorder is difficult to identify non-invasively, and because its pathogenesis is not well understood, effective rational therapies are lacking.

There is a need in the art for methods of treating TNF-α-mediated disorders. The present invention addresses this need.

### Literature

U.S. Pat. No. 5,962,478; U.S. Pat. No.6,025,151; U.S. Pat. No. 6,300,349; U.S. Pat. No. 6,294,350; Antoni and Braun (2002) Rheumatol. 20:S152-S157; and Bresnihan and Cunnane (2003) Rheum. Dis. Clin. North Am. 29:185-202; Pedersen et al. (2003) Mech. Ageing Dev. 124:495-502; Holstad and Sandler (2001) Autoimmun 16:441-447; Moller (2000) Trends Endocrinol. Metab. 11:212-217.

### SUMMARY OF THE INVENTION

The present invention provides methods of treating TNF-α-mediated disorders, the methods generally involving administering to an individual in need thereof effective amounts of pirfenidone or a pirfenidone analog and a second therapeutic agent that reduces TNF-α synthesis or that reduces TNF-α binding to a TNF receptor. The present invention further provides methods for treating non-alcoholic steatohepatitis, the method generally involving administering to an individual in need thereof an effective amount of pirfenidone. The present invention further provides methods of treating end-stage or advanced Type II diabetes, the methods generally involving administering to an individual in need thereof effective amounts of pirfenidone and insulin.

### BRIEF DESCRIPTION OF THE DRAWINGS

FIG. 1 depicts insulin signaling in the absence of TNF.

FIG. 2 depicts insulin signaling in the presence of high concentrations of TNF.

FIG. 3 depicts inhibition of MAPK by pirfenidone.

FIG. 4 depicts enhancement of insulin signaling by pirfenidone in the presence or absence of TNF.

FIG. 5 depicts various downstream signaling events that are triggered by TNF binding to a TNF receptor.

FIG. 6 depicts pirfenidone inhibition of TNF-induced ERK activation.

FIG. 7 depicts pirfenidone inhibition of TNF-induced p38 MAPK activation.

FIG. 8 depicts pirfenidone inhibition of TNF-induced activation of the transcription factor CREB.

FIG. 9 depicts pirfenidone inhibition of TNF-induced activation of RAF kinase.

FIG. 10 depicts pirfenidone inhibition of TNF-induced activation of AKT.

FIG. 11 depicts pirfenidone potentiation of TNF-induced JNK activation.

FIG. 12 depicts pirfenidone potentiation of ATF2.

### DEFINITIONS

As used herein, the terms “treatment,” “treating,” and the like, refer to obtaining a desired pharmacologic and/or physiologic effect. The effect may be prophylactic in terms of completely or partially preventing a disease or symptom thereof and/or may be therapeutic in terms of a partial or complete cure for a disease and/or adverse affect attributable to the disease. “Treatment,” as used herein, covers any treatment of a disease in a mammal, particularly in a human, and includes: (a) increasing survival time; (b) decreasing the risk of death due to the disease; (c) preventing the disease from occurring in a subject which may be predisposed to the disease but has not yet been diagnosed as having it; (d) inhibiting the disease, i.e., arresting its development (e.g., reducing the rate of disease progression); and (e) relieving the disease, i.e., causing regression of the disease.

A “specific pirfenidone analog,” and all grammatical variants thereof, refers to, and is limited to, each and every pirfenidone analog shown in Table 1.

The terms “individual, ” “host,” “subject,” and “patient,” used interchangeably herein, refer to a mammal, particularly a human.

Before the present invention is further described, it is to be understood that this invention is not limited to particular embodiments described, as such may, of course, vary. It is also to be understood that the terminology used herein is for the purpose of describing particular embodiments only, and is not intended to be limiting, since the scope of the present invention will be limited only by the appended claims.

Where a range of values is provided, it is understood that each intervening value, to the tenth of the unit of the lower limit unless the context clearly dictates otherwise, between the upper and lower limit of that range and any other stated or intervening value in that stated range, is encompassed within the invention. The upper and lower limits of these smaller ranges may independently be included in the smaller ranges, and are also encompassed within the invention, subject to any specifically excluded limit in the stated range. Where the stated range includes one or both of the limits, ranges excluding either or both of those included limits are also included in the invention.

Unless defined otherwise, all technical and scientific terms used herein have the same meaning as commonly understood by one of ordinary skill in the art to which this invention belongs. Although any methods and materials similar or equivalent to those described herein can also be used in the practice or testing of the present invention, the preferred methods and materials are now described. All publications mentioned herein are incorporated herein by reference to disclose and describe the methods and/or materials in connection with which the publications are cited.

It must be noted that as used herein and in the appended claims, the singular forms “a”, “and”, and “the” include plural referents unless the context clearly dictates otherwise. Thus, for example, reference to “a TNF-α antagonist” includes a plurality of such antagonists and reference to “the agent” includes reference to one or more agents and equivalents thereof known to those skilled in the art, and so forth.

The publications discussed herein are provided solely for their disclosure prior to the filing date of the present application. Nothing herein is to be construed as an admission that the present invention is not entitled to antedate such publication by virtue of prior invention. Further, the dates of publication provided may be different from the actual publication dates which may need to be independently confirmed.

### DETAILED DESCRIPTION OF THE INVENTION

The present invention provides methods of treating TNF-α-mediated disorders, the methods generally involving administering to an individual in need thereof an effective amount of pirfenidone or a pirfenidone analog and a sub-therapeutic amount of a second therapeutic agent, such as a TNF-α antagonist that reduces TNF-α synthesis or that reduces TNF-α binding to a TNF receptor.

Currently, a number of TNF-mediated disorders are treated with TNF-α antagonists such as ENBREL® and REMICADE®. However, long-term systemic exposure to TNF-α antagonists such as ENBREL® and REMICADE® can impose a risk for increased viral and bacterial infections. Antoni and Braun (2002) Rheumatol. 20:S152-S157; and Bresnihan and Cunnane (2003) Rheun. Dis. Clin. North Am. 29:185-202.

The present methods for treating TNF-α-mediated disorders involve administering both pirfenidone or a pirfenidone analog and another TNF-α antagonist. The subject methods are advantageous in that lower doses of the TNF-α antagonist are administered, thereby reducing the risk of increased viral and bacterial infections currently associated with use of such antagonists. Without intending to be limited to any particular theory, it is believed that the pleiotropic effect of pirfenidone on transcription activating factors AP-1 and CREB permits the creation of an intermediate level of transcription of genes controlled by TNF-induced promoters.

Pirfenidone inhibits CREB activation and potentiates AP-1 activation. Moreover, even at maximal pirfenidone concentrations the inhibition of CREB activation does not exceed an upper limit of approximately 75%. Thus, at high concentrations of pirfenidone a cell system can still retain the potential to activate CREB up to about 25% of the maximum level of CREB activation. In addition, some TNF-inducible promoters are regulated by activated CREB/activated AP-1 complex and can exhibit intermediate levels of transcription induction. Whereas the modulation of this promoter system with an antagonist of TNF binding to TNFR (e.g. REMICADE) is constrained by the kinetics of AP-1/CREB/promoter interaction in a native intracellular environment, the modulation of this promoter system with a combination of pirfenidone and an antagonist of TNF binding to TNFR allows the clinician to achieve an intermediate state of transcription activity beyond the lower limit imposed by the kinetics of the native intracellular environment. The higher level of activated AP-1 achieved in the system with pirfenidone supports complex formation with the remaining (approximately 25%) activated CREB, and consequent binding to promoter and initiation of transcription, at a higher frequency than that supported by the lower levels of activated AP-1 achieved in the system with only an antagonist of TNF binding to TNFR (without pirfenidone).

The present invention further provides methods for treating non-alcoholic steatohepatitis (NASH), the method generally involving administering to an individual in need thereof an effective amount of pirfenidone. NASH is increasingly recognized as a relatively prevalent disorder that can lead to cirrhosis in some individuals. In fact, nearly 20% of patients with histologically proven NASH progress to cirrhosis. NASH can also progress to hepatic insufficiency and hepatocellular carcinoma. Because this disorder is difficult to identify non-invasively, and because its pathogenesis is not well understood, effective rational therapies are lacking. Risk factors for NASH include obesity. TNF-α-induced insulin resistance is a common feature of obesity.

The present invention further provides methods of treating end-stage or advanced Type II diabetes, the methods generally involving administering to an individual in need thereof effective amounts of pirfenidone and insulin. The progression of Type II diabetes is as follows. Early stages of Type II diabetes are characterized insulin resistance; although insulin is produced in normal amounts, and can bind normally to receptors on liver and muscle cells, blood glucose is not imported into the cells. Most Type II diabetics produce variable, even normal or high, amounts of insulin, and in the beginning this amount is usually sufficient to overcome such resistance. Over time, the pancreas becomes unable to produce enough insulin to overcome resistance. In Type II diabetes the initial effect of this stage is usually an abnormal rise in blood sugar right after a meal (“postprandial hyperglycemia”). This effect is believed to be particularly damaging to the body. Eventually, the cycle of elevated glucose further impairs and possibly destroys the pancreatic beta cells that produce insulin, thereby stopping insulin production completely and causing full-blown diabetes. This is made evident by fasting hyperglycemia, in which elevated glucose levels are present most of the time.

The subject methods of treating Type II diabetes are advantageous in that they provide for reduction of the deleterious effects of TNF-α in insulin resistance; and provides insulin, which is deficient in patients with advanced stage Type II diabetes.

### Therapeutic Methods

The present invention provides a method of treating a TNF-α-mediated disorder, the method generally involving administering to an individual in need thereof an effective amount of pirfenidone or a pirfenidone analog, and a sub-therapeutic amount of a second therapeutic agent that inhibits TNF-α synthesis or that inhibits binding of TNF-α to a TNF receptor. The present invention further provides a method of treating non-alcoholic steatohepatitis (NASH), the method generally involving administering to an individual in need thereof an effective amount of pirfenidone or a pirfenidone analog. The present invention further provides a method of treating advanced Type II diabetes, the method generally involving administering to an individual in need thereof an effective amount of pirfenidone or a pirfenidone analog and an effective amount of insulin.

### Treatment of TNF-Mediated Disorders

The present invention provides a method of treating a TNF-α-mediated disorder. The method generally involves administering to the patient a sub-therapeutic amount of a non-pirfenidone compound (e.g., a compound other than pirfenidone or a pirfenidone analog) that is lower than a minimum dose of the compound that, when administered in the absence of concomitant pirfenidone or pirfenidone analog therapy, is effective to reduce one or more signs or symptoms of the disorder in the patient; and co-administering to the patient an effective amount of pirfenidone or a pirfenidone analog that, in combination with the sub-therapeutic amount of the non-pirfenidone compound, is effective to reduce one or more signs or symptoms of the TNF-mediated disorder in the patient, while reducing or avoiding the severity or incidence of infection that would arise from the use of the non-pirfenidone compound at the minimum dose or greater. In some embodiments, the method generally involving administering to an individual in need thereof an effective amount of pirfenidone or a pirfenidone analog, and a sub-therapeutic amount of a second therapeutic agent, e.g., a TNF-α antagonist, wherein the TNF-α antagonist is an agent that inhibits TNF synthesis or that inhibits binding of TNF to a TNF receptor.

An “effective amount” of pirfenidone or a pirfenidone analog, and a “sub-therapeutic amount” of a non-pirfenidone compound (a TNF-α antagonist) are defined as follows. An “sub-therapeutic amount” of a non-pirfenidone compound, e.g., a TNF-α antagonist, is an amount that is lower than the minimum dose of the TNF-α antagonist compound that, when administered free of concomitant therapy with pirfenidone or pirfenidone analog, is effective to reduce one or more of the signs or symptoms of a TNF-α-mediated disorder. Although the “sub-therapeutic amount” of the non-pirfenidone compound would not be effective, free of concomitant therapy with pirfenidone or pirfenidone analog, to reduce one or more of the signs or symptoms of a TNF-α-mediated disorder, when administered together with an effective amount of pirfenidone or pirfenidone analog, the “sub-therapeutic amount” of the non-pirfenidone compound (e.g., the TNF-α antagonist) is effective to reduce one or more of the signs or symptoms of a TNF-α-mediated disorder.

In general, a sub-therapeutic amount of a non-pirfenidone compound is an amount that is from about 10% to about 95% less than the minimum dose that, when administered free of concomitant therapy with pirfenidone or pirfenidone analog, is effective to reduce one or more of the signs or symptoms of a TNF-α-mediated disorder. For example, an effective amount of a non-pirfenidone compound is an amount that is from about 10% to about 15%, from about 15% to about 20%, from about 20% to about 25%, from about 25% to about 30%, from about 30% to about 35%, from about 35% to about 40%, from about 40% to about 45%, from about 45% to about 50%, from about 50% to about 55%, from about 55% to about 60%, from about 60% to about 65%, from about 65% to about 70%, from about 70% to about 75%, from about 75% to about 80%, from about 80% to about 85%, from about 85% to about 90%, or from about 90% to about 95% lower than the minimum dose that, when administered free of concomitant therapy with pirfenidone or pirfenidone analog, is effective to reduce one or more of the signs or symptoms of a TNF-α-mediated disorder. In some embodiments, a sub-therapeutic amount of a non-pirfenidone compound is an amount that is from about 10% to about 15%, from about 15% to about 20%, from about 20% to about 25%, from about 25% to about 30%, from about 30% to about 35%, from about 35% to about 40%, from about 40% to about 45%, from about 45% to about 50%, from about 50% to about 55%, from about 55% to about 60%, from about 60% to about 65%, from about 65% to about 70%, from about 70% to about 75%, from about 75% to about 80%, from about 80% to about 85%, from about 85% to about 90%, or from about 90% to about 95% lower than the amount that is typically prescribed for the compound.

In some embodiments, sub-therapeutic amounts of a non-pirfenidone compound are amounts that, when administered in combination therapy with an effective amount of pirfenidone or pirfenidone analog, are effective to reduce a sign or symptom of a TNF-mediated disorder by at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, or at least about 80% or more, when compared to the level or severity of the sign or symptom in the individual not treated with the combination therapy.

Signs or symptoms associated with rheumatoid arthritis include erythrocyte sedimentation rate (ESR); joint pain; swollen joints; joint damage; and the like. Joint damage can be assessed radiographically and expressed as change in the Total Sharp Score (TSS), and its components, the erosion score and the Joint Space Narrowing (JSN) score.

For example, where the TNF-α-mediated disorder is rheumatoid arthritis, sub-therapeutic amounts of a non-pirfenidone compound and effective amounts of pirfenidone or a pirfenidone analog are amounts effective to reduce the degree of joint swelling and/or joint tenderness by at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, or at least about 80% or more, when compared to the degree or severity of joint swelling or tenderness in the individual not treated with the combination therapy.

In some embodiments, where the TNF-α-mediated disorder is rheumatoid arthritis, sub-therapeutic amounts of a non-pirfenidone compound and effective amounts of pirfenidone or a pirfenidone analog are amounts effective to achieve a 20% or greater improvement in 4 of the following: (1) tender and swollen joint counts; (2) morning stiffness; (3) patient assessment of disease activity; (4) physician assessment of disease activity; and (5) erythrocyte sedimentation rate (ESR).

In some embodiments, where the TNF-α-mediated disorder is rheumatoid arthritis, sub-therapeutic amounts of a non-pirfenidone compound and effective amounts of pirfenidone or a pirfenidone analog are amounts effective to achieve ACR 20, ACR 50, or ACR 70, e.g., a 20%, 50%, or 70% improvement in tender and swollen joint counts and improvement in three of the following parameters: (1) physician assessment; (2) ESR; (3) pain scale; and (4) functional questionnaire. ACR 20, ACR 50, and ACR 70 are American College of Rheumatology criteria for improvement of rheumatoid arthritis symptoms. See, e.g., Arthritis and Rheum. 46(2):328 (2002).

Where the TNF-α-mediated disorder is an inflammatory bowel disease such as Crohn's disease or ulcerative colitis, sub-therapeutic amounts of a non-pirfenidone compound and effective amounts of pirfenidone or a pirfenidone analog are amounts effective to reduce one or more of the following symptoms: cramping; abdominal pain; diarrhea; fever; weight loss; bloating; anal pain or drainage; skin lesions; rectal abscess; intestinal ulcers; fissure; and joint pain. Sub-therapeutic amounts of a non-pirfenidone compound and effective amounts of pirfenidone or a pirfenidone analog are amounts effective to reduce one or more of the foregoing symptoms by at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, or at least about 80% or more, when compared to the degree or severity of the symptom in the individual not treated with the combination therapy.

In some embodiments, a subject method involves administering a synergistic combination of pirfenidone or a pirfenidone analog and a TNF-α antagonist. As used herein, a “synergistic combination” of pirfenidone or a pirfenidone analog and a TNF-α antagonist is a combined dosage that is more effective in the therapeutic or prophylactic treatment of a TNF-α-mediated disorder than the incremental improvement in treatment outcome that could be predicted or expected from a merely additive combination of (i) the therapeutic or prophylactic benefit of pirfenidone or a pirfenidone analog when administered at that same dosage free of concomitant therapy with the TNF-α antagonist and (ii) the therapeutic or prophylactic benefit of the TNF-α antagonist when administered at the same dosage free of concomitant therapy with pirfenidone or pirfenidone analog.

TNF-α-Mediated Disorders

A subject method for treating a TNF-α-mediated disorder is useful for treating any TNF-mediated disorder. The term “TNF-α-mediated disorder” refers to any disorder or disease state in which TNF-α plays a direct role, e.g., by excessive production or release of TNF-α itself or by TNF-α-induced production or release of another agent that produces a pathological effect. As such, a subject method for treating a TNF-α-mediated disorder is useful for treating any fibrotic disorder, including obliterative bronchiolitis, interstitial lung disease, fibrotic lung disease (e.g., idiopathic pulmonary fibrosis (IPF), pulmonary fibrosis of a known etiology, e.g. cystic fibrosis, adult respiratory distress, syndrome (ARDS), tumor stroma in lung disease, systemic sclerosis, Hermansky-Pudlak syndrome (HPS), coal worker's pneumoconiosis (CWP), asbestosis, sarcoidosis, silicosis, black lung disease, chronic pulmonary hypertension, AIDS associated pulmonary hypertension, and the like), human kidney disease (e.g., nephrotic syndrome, Alport's syndrome, HIV-associated nephropathy, polycystic kidney disease, Fabry's disease, diabetic nephropathy, and the like), glomerular nephritis, nephritis associated with systemic lupus erythematosus, fibrotic vascular disease, arterial sclerosis, atherosclerosis, varicose veins, coronary infarcts, cerebral infarcts, musculoskeletal fibrosis, post-surgical adhesions, cutis keloid formation, progressive systemic sclerosis, primary sclerosing cholangitis (PSC), renal fibrosis, scleroderma (local and systemic), diabetic retinopathy, glaucoma, Peyronie's disease, penis fibrosis, arethrostenosis after test using cystoscope, inner accretion after surgery, myelofibrosis, idiopathic retroperitoneal fibrosis, fibrosis incident to microbial infection (e.g. viral, bacterial, fungal, parasitic, etc.), fibrosis incident to inflammatory bowel disease (including stricture formation in Crohn's disease and microscopic colitis), fibrosis induced by chemical or environmental insult (e.g., cancer chemotherapy, pesticides, radiation (e.g. cancer radiotherapy), and the like), peritoneal fibrosis, liver fibrosis, myocardial fibrosis, pulmonary fibrosis, Grave's ophthalmopathy, drug induced ergotism, cardiovascular disease, fibrosis incident to benign or malignant cancer (including desmoid tumor), Alzheimer's disease, scarring, scleroderma, glioblastoma in Li-Fraumeni syndrome, sporadic glioblastoma, myeloid leukemia, acute myelogenous leukemia, myelodysplastic syndrome, myeloproliferative syndrome, fibrosis incident to benign or malignant gynecological cancer (e.g., ovarian cancer, Lynch syndrome, and the like), Kaposi's sarcoma, Hansen's disease, inflammatory bowel disease (including stricture formation in Crohn's disease and microscopic colitis), Crohn's disease, ulcerative colitis, multiple sclerosis, Type II diabetes, rheumatoid arthritis, asthma, chronic bronchitis, atopic dermatitis, urticaria, allergic rhinitis, allergic conjunctivitis, chronic obstructive pulmonary disease, graft rejection, graft-versus-host disease, sepsis, and the like.

CNS disorders Evidence exists in the literature that TNF-α has effects on cells of the central nervous systems (CNS). Evidence for CNS production of TNF-α, involvement of TNF-α in brain injury, the role of polymorphonuclear leukocytes (PMNs) in brain injury, the role of adhesion molecules in brain injury, and potential TNF-α directed therapeutic strategies for prevention of brain injury have been reviewed in the literature. See, e.g., Babak Arvin et al. (1995) Ann. N.Y. Acad. Sciences 765: 62-71.

The prevention of brain edema by anti-TNF-α antibodies in experimental meningitis provides firm evidence for the involvement of TNF-α in the breakdown of the Blood Brain Barrier. TNF-α can also trigger the infiltration of neutrophils into the tissue with consequent induction of secondary mediators in local areas. See, e.g., “Cytokines and CNS,” Edit: R. M. Ransohoff and E. N. Beneviste, CRC Press, Page 193, 1996).

Closed head injury (CHI) in rats triggers the production of TNF-α in the contused brain hemisphere, and it was shown that a decrease in TNF-α levels or inhibition of its activity is accompanied by significantly reduced brain damage. Shohami et al. (1996) J. Cerebral Blood Flow Metab., 16:378-384.

Multiple Sclerosis Multiple sclerosis (MS) plaques within the CNS are infiltrated by peripheral blood mononuclear cells. In patients, TNF-α, but not lymphotoxin, is overproduced by peripheral blood mononuclear cells during MS relapse. Glabinski et al. (1995) Neurol Scand. 91:276-279. TNF-α has an ability to cause cell death of oligodendrocytes in vitro. Robbins et al. (1987) J. Immunol., 139:2593. This aspect of TNF-α activity may contribute directly to myelin damage and/or the demyelination process observed in diseases such as multiple sclerosis (MS). TNF-α has been shown to play a central role in the demyelination of the CNS in MS. Serum levels of TNF-α are elevated in patients with active MS, and TNF-αproducing macrophages, microglia and astrocytes are present at active lesion sites. In in vitro experiments, TNF-α directly mediates oligodendrocyte damage and suppresses myelin formation, and it stimulates astrocytes, which are then responsible for the CNS scarring plaques in MS (Owens and Sriram, Neurological Clinics, 13:51, 1995).

Serum levels of TNFα are elevated in patients with active MS (M. Chofflon et al., Eur. Cytokine Net., 3:523, 1991; Sharief, M. K. and Hentgen, N. E. Jour. Med., 325:467, 1991). TNF-α producing macrophages/microglia and astrocytes are present at active lesion sites (K. Selmaj al., Jour. Clin. Invest., 87:949, 1991). In in vitro experiments, TNF-α directly mediates oligodendrocyte damage and suppresses myelin formation (K. Selmaj et al., J. Immunol., 147:1522, 1990); T. Tsumamoto et al., Acta Neurol. Scand., 91:71, 1995), and it stimulates astrocytes, which are responsible for the scarring plaques (K. Selmaj et al., J. Immunol., 144:129, 1990).

An increase in TNF-α expression preceding MS exacerbation attacks has been shown. (“Cytokines and the CNS,” Edit: R. M. Ransohoff and E. N. Beneviste, CRC Press, 1996, p. 232). In vivo studies of murine, rat and human demyelinating diseases indicate that TNF-α participates in the inflammatory reactions that take place within the CNS. TNF-α positive astrocytes and macrophages have been identified in the brains of MS patients, particularly in the plaque region (F. M. Hofman et al., J. Exp. Med., 170:607, 1991, and Selmaj et al., J. Clin. Invest., 87:949, 1991) have determined that both TNF-α and TNF-β are present in MS plaque regions, and that TNF-α is localized within astroyctes, whereas TNF-α is associated with microglia and T-cells. Increased serum and cerebrospinal fluid levels of TNF-α have been documented in patients with MS (Sharief, M. K., M. Phil, and R. Hentges, N. Engl. J. Med., 325:467, 1991), and a strong correlation exists between cerebrospinal fluid levels of TNF-α disruption of the blood brain barrier, and high levels of circulating ICAM-1 in patients with active MS.

Alzheimer's Disease Alzheimer's disease (AD), the most common dementing disorder of late life, is a major cause of disability and death in the elderly. The disease is manifested by the appearance of abnormalities in the brain, particularly involving the hippocampus, amygdala, thalamus and neocortex. Lesions in these regions are associated with dysfunction/death of neurons and deafferentation of targets. The principal pathological hallmarks of AD are deposits of the amyloid-β protein (Aβ) in extracellular parenchyma and cerebral vessels, and neurofibrillary tangles.

TNF-α has been generally elevated in the serum of AD patients based upon both antibody assays and bioassays. In one study almost half of the AD cases had elevated TNFα, but none of the controls had a similar elevation. The blood-brain barrier does not normally permit passage of cytokines. However, there is evidence to suggest that the blood-brain barrier may not be intact in AD.

Respiratory disorders TNF-α has been shown to play a role in pulmonary fibrosis induced by bleomycin and silica (Piguet et al., Jour. Exper. Med., 170:655-663, 1989, and Nature, 344:245-247, 1990; Everson and Chandler, Amer. Jour. Path., 140:503-512, 1992; Phan and Kunkel, Exp. Lung Res. 18:2943, 1992; also, Warren et al., Jour. Clin. Invest., 84:1873-1882, 1989; Denis et al., Amer. Jour. Cell Mol. Biol., 5:477-483, 1991). TNF-α has been reported to orchestrate its proinflammatory effects by regulating the compartmentalized release of secondary messenger cytokines. Investigations have shown that nude mice exposed to chronic in vivo TNF-α develop pulmonary inflammation and fibrosis (ARRD 145:A307, 1992).

Asthma It has been reported that levels of TNF-α are increased in bronchoalveolar lavage (BAL) fluid from patients with allergic asthma. Cirelli, et al. (1995) Amer. Jour. Resp. Critical Care Med., 151:345A; Redington et al., (1995) Amer. Jour. Respir. Crit. Care Med., 151: 702A. These findings indicate an increased tissue level of TNF-α in asthma and that this may contribute to the pathophysiology of the condition.

Chronic Obstructive Pulmonary Disease (COPD) Another disease state in which TNF-α plays a role in the pathophysiology is chronic obstructive pulmonary disease. In silicosis, a disease of progressive respiratory failure caused by a fibrotic reaction, antibody to TNF-α completely blocked the silica-induced lung fibrosis in mice (Piguet et al., Nature, 344:245-247, 1990). High levels of TNF production (in the serum and in isolated macrophages) have been demonstrated in animal models of silica and asbestos induced fibrosis (Bissonnette et al., Inflammation, 13:329-339, 1989).

Adult Respiratory Distress Syndrome (ARDS) Excessive TNF-alpha. concentrations in excess of 12,000 pg/ml have been detected in pulmonary aspirates from ARDS patients (Millar et al., Lancet, 2(8665):712-714, 1989). Systemic infusion of recombinant TNF-α was shown to result in changes typically seen in ARDS (Ferrai-Baliviera et al., Arch. Surg., 124:1400-1405, 1989).

Lung Sarcoidosis Alveolar macrophages from pulmonary sarcoidosis patients have been found to spontaneously release massive quantities of TNF-α as compared with macrophages from normal donors (Baughman et al., Jour. Lab. Clin. Med., 115:36-42, 1990). TNF-α also implicated in other acute disease states such as the pathophysiologic responses which follows subsequent reperfusion. It is involved in reperfusion injury, and is a major cause of tissue damage after loss of blood flow. (Vedder et al., Proc. Nat. Acad. Sci., 87:2643-2646, 1990).

Sepsis Overproduction of TNF-α has been implicated in the pathogenesis of endotoxin induced septic shock, (see Carswell et al., Proc. Nat. Acad. Sci., 2:3666-3670, 1975). Endotoxin is the lipopolysaccharide component of the cell wall of gram-negative bacteria, and is a macrophage activator which induces the synthesis and enhanced secretion of TNF-α and other biologically active cytokine molecules. TNF-α is recognized as a central mediator of sepsis, septic shock and multiple organ failure. These host reactions are associated with increased blood levels of TNF-α, due to increased TNF-α production. (F. Stuber et al., Jour. Inflam., 46:42-50, 1996).

Liver disorders Because of its central role in metabolism and host defense mechanisms, the liver is thought to be major organ responsible for initiation of the multiple organ failure during sepsis. The depression in hepatocellular function in early, hyperdynamic stages of sepsis does not appear to be due to any reduction in hepatic perfusion, but is associated with elevated levels of circulating cytokines such as TNF-α. Furthermore, administration of recombinant TNF-α at doses that do not reduce cardiac output or hepatic perfusion, produces hepatocellular dysfunction. (P. Wang et al., Amer. Jour. Physiol., 270:5, 1996).

The role of TNF-α in induction of hepatic apoptosis under transcriptional arrest, activation of the 55 kDa receptor in the induction of hepatic apoptosis, the glycosylation step in TNF-induced hepatic apoptosis, hepatic injury induction by T cell-initiated cytokine release, and T cell-dependent TNF-mediated liver injury without transcriptional arrest has been reported. A. Wendel et al., Cell. Biol. Mol. Basis Liver Transp., Int., Ringberg Conf. Hepatic Transp., 2nd, 1995, Pages 105-111; and Leist et al. Am J Pathol. 1995 May; 146(5):1220-34.

Diabetes TNF-α plays a central role in the state of insulin resistance associated with obesity. It has been previously shown that one important mechanism by which TNF-α interferes with insulin signaling is through serine phosphorylation of insulin receptor substrate-1 (IRS-1), which can function as an inhibitor of the tyrosine kinase activity of the insulin receptor (IR). The data strongly suggest that TNF-α inhibits signaling via a stimulation of p55 TNFR, and sphingomyelinase activity, which results in the production of an inhibitory form of IRS-1 (Peraldi et al., J. Biol. Chem. 271:13018-13022, 1996).

Crohn's disease TNF-α levels are elevated in Crohn's disease. In one study, TNF-α concentrations were measured in stool samples from normal children, infants with diarrhea, and children with inflammatory bowel disease in active and inactive phases. Compared with diarrhea controls, stool TNF-α concentrations were significantly increased in children with active Crohn's disease. In patients with inactive Crohn's disease, either as a result of surgery, or treatment with steroids, the concentration of stool TNF-α fell to the level of the controls (C. P. Braegger et al., Lancet, 339:89-91, 1992).

Pre-Eclampsia Pre-eclampsia is an endothelial disorder, and TNF-α has fundamental effects on endothelial cells by several means, including alteration of the balance between oxidant and anti-oxidant, changing the pattern of prostaglandin production, and affecting the expression of several cell surface components. In patients, results show that TNF-α mRNA expression is significantly elevated in preeclamptic patients compared to the control groups. These observations are consistent with a major role for TNF-α in the development of eclampsia (G. Chen et al., Clin. Exp. Immunol. 104:154-159, 1996).

Dermal Burns The protein catabolic rate and TNF-α content of the soleus muscle of the scalded region and remote region were dynamically determined in the first week after the rats were inflicted with 37% total body surface area (TBSA) full thickness scalding. The TNF-α content of skeletal muscles was far greater in the scalded region than in the remote region. TNF-α increase was also significantly correlated to the protein catabolic rate of the skeletal muscles (Li et al., Jour. Med. Coll., PLA 10:262-267, 1995; C.A. 125:938, 1245:8156a, 1996).

Bone Resorption TNF-α is increased in bone resorption diseases, including arthritis, wherein it has been determined that when activated, leukocytes will produce a bone reabsorbing activity. Data indicate that TNF-α enhances this activity (Bertolini et al., Nature, 319:516-518, 1986, and Johnson et al., Endocrinology, 124:1424-1427, 1989). TNF-α stimulates bone resorption and inhibits bone formation in vitro and in vivo through stimulation of osteoclast formation and activation combined with inhibition of osteoblast function. TNF-α may be involved in many bone resorption diseases, including arthritis.

Rheumatoid Arthritis Analysis of cytokine mRNA and protein in human rheumatoid arthritis tissue revealed that many proinflammatory cytokines such as TNF-α are abundant in all patients regardless of therapy. In rheumatoid joint cell cultures that spontaneously produce IL1, TNF-α was the major dominant regulator of IL1. Subsequently, other proinflammatory cytokines were also inhibited if TNF-α was neutralized, leading to the concept that the proinflammatory cytokines were linked in a network with TNF-α at its apex. This led to the concept that TNF-α was of major importance in rheumatoid arthritis. This has been successfully tested in animal models of collagen-induced arthritis, and these studies have provided the rationale for clinical trials of anti-TNF-α therapy in patients with long-standing rheumatoid arthritis. Several clinical trials using a chimeric anti-TNF-α antibody have shown marked clinical benefit, verifying the concept that TNF-α is of major importance in rheumatoid arthritis. Re-treatment clinical studies have also shown benefit in repeated relapses, indicating that the disease remains TNF-α dependent (M. Feldmann, Annual Rev. Immunol., 14:397-440, 1996.).

Vascular disorders TNF-α alters the properties of endothelial cells and has various pro-coagulant activities, such as production of an increase in tissue factor procoagulant activity and suppression of the anticoagulant protein C pathway as well as down-regulating the expression of thrombomodulin (Sherry et al., Jour. Cell. Biol., 107:1269-1277, 1988). TNF-α has activities which, together with its early production (during the initial stages of a trauma or injury event), make it a mediator of response to tissue injury in several important disorders including, but not limited to myocardial infarction, stroke and circulatory shock. Of specific importance may be INF-α induced expression of adhesion molecules, such as intercellular adhesion molecule (ICAM) or endothelial leukocyte adhesion molecule on endothelial cells (Munro et al., Am. Jour. Path., 135:121-132, 1989).

Cardiac disorders Evidence indicates that the current top suspects in heart failure are noradrenaline, angiotensin, vasopressin, endothelin, and tumor-necrosis factor (TNF-.alpha.), (N.E. J. Med., 323:236-241, 1990). It has been reported that concentrations of TNF-α, which cause cachexia in chronic inflammatory disorders, infections, cancer and other diseases, are elevated in patients with severe heart failure, especially those with the more severe manifestations of the disease, such as cardiac cachexia.

Graft vs. host disease In graft versus host reactions, increased serum TNF-α levels have been associated with major complications following acute allogeneic bone marrow transplants (Holler et al., Blood, 75:1011-1016, 1990).

### Non-Pirfenidone Agents

Suitable second, non-pirfenidone therapeutic agents include agents that decrease the level of TNF-α synthesis; and agents that block binding of TNF-α to a TNF-α receptor (TNFR). Agents that decrease the level of TNF-α synthesis, and agents that block binding of TNF-α to a TNF receptor are collectively referred to herein as TNF-α antagonists. Agents that block binding of TNF-α to TNFR include soluble TNFR, antibody to TNF-α, and the like.

As used herein, the terms “TNF receptor polypeptide” and “TNFR polypeptide” refer to polypeptides derived from TNFR (from any species) which are capable of binding TNF. Two distinct cell-surface TNFRs have described: Type II TNFR (or p75 TNFR or TNFRII) and Type I TNFR (or p55 TNFR or TNFRI). The mature full-length human p75 TNFR is a glycoprotein having a molecular weight of about 75-80 kilodaltons (kD). The mature full-length human p55 TNFR is a glycoprotein having a molecular weight of about 55-60 kD. Exemplary TNFR polypeptides are derived from TNFR Type I and/or TNFR type II. Soluble TNFR includes p75 TNFR polypeptide; fusions of p75 TNFR with heterologous fusion partners, e.g., the Fc portion of an immunoglobulin.

TNFR polypeptide may be an intact TNFR or a suitable fragment of TNFR. U.S. Pat. No. 5,605,690 provides examples of TNFR polypeptides, including soluble TNFR polypeptides, appropriate for use in the present invention. In many embodiments, the TNFR polypeptide comprises an extracellular domain of TNFR. In some embodiments, the TNFR polypeptide is a fusion polypeptide comprising an extracellular domain of TNFR linked to a constant domain of an immunoglobulin molecule. In other embodiments, the TNFR polypeptide is a fusion polypeptide comprising an extracellular domain of the p75 TNFR linked to a constant domain of an IgG1 molecule. In some embodiments, when administration to humans is contemplated, an Ig used for fusion proteins is human, e.g., human IgG1.

Monovalent and multivalent forms of TNFR polypeptides may be used in the present invention. Multivalent forms of TNFR polypeptides possess more than one TNF binding site. In some embodiments, the TNFR is a bivalent, or dimeric, form of TNFR. For example, as described in U.S. Pat. No. 5,605,690 and in Mohler et al., 1993, J. Immunol., 151:1548-1561, a chimeric antibody polypeptide with TNFR extracellular domains substituted for the variable domains of either or both of the immunoglobulin heavy or light chains would provide a TNFR polypeptide for the present invention. Generally, when such a chimeric TNFR:antibody polypeptide is produced by cells, it forms a bivalent molecule through disulfide linkages between the immunoglobulin domains. Such a chimeric TNFR:antibody polypeptide is referred to as TNFR:Fc.

In one embodiment, a subject method involves administration of an effective amount of the soluble TNFR ENBREL®. ENBREL® is a dimeric fusion protein consisting of the extracellular ligand-binding portion of the human 75 kilodalton (p75) TNFR linked to the Fc portion of human IgG1. The Fc component of ENBREL® contains the CH2 domain, the CH3 domain and hinge region, but not the CH1 domain of IgG1. ENBREL® is produced in a Chinese hamster ovary (CHO) mammalian cell expression system. It consists of 934 amino acids and has an apparent molecular weight of approximately 150 kilodaltons. Smith et al. (1990) Science 248:1019-1023; Mohler et al. (1993) J. Immunol. 151:1548-1561; U.S. Pat. No. 5,395,760; and U.S. Pat. No. 5,605,690.

Also suitable for use are monoclonal antibodies that bind TNF-α. Monoclonal antibodies include “humanized” mouse monoclonal antibodies; chimeric antibodies; monoclonal antibodies that are at least about 80%, at least about 90%, at least about 95%, or 100% human in amino acid sequence; and the like. See, e.g., WO 90/10077; WO 90/04036; and WO 92/02190. Suitable monoclonal antibodies include antibody fragments, such as Fv, F(ab′)₂ and Fab; synthetic antibodies; artificial antibodies; phage display antibodies; and the like.

Examples of suitable monoclonal antibodies include Infliximab (REMICADE®, Centocor); and Adalimumab (HUMIRA™, Abbott). REMICADE® is a chimeric monoclonal anti-TNF-α antibody that includes about 25% mouse amino acid sequence and about 75% human amino acid sequence. REMICADE® comprises a variable region of a mouse monoclonal anti-TNF-α antibody fused to the constant region of a human IgG1. Elliott et al. (1993) Arthritis Rheum. 36:1681-1690; Elliott et al. (1994) Lancet 344:1105-1110; Baert et al. (1999) Gastroenterology 116:22-28. HUMIRA™ is a human, full-length IgG1 monoclonal antibody that was identified using phage display technology. Piascik (2003) J. Am. Pharm. Assoc. 43:327-328.

Methods to assess TNF antagonist activity are known in the art and exemplified herein. For example, TNF antagonist activity may be assessed with a cell-based competitive binding assay. In such an assay, radiolabeled TNF is mixed with serially diluted TNF antagonist and cells expressing cell membrane bound TNFR. Portions of the suspension are centrifuged to separate free and bound TNF and the amount of radioactivity in the free and bound fractions determined. TNF antagonist activity is assessed by inhibition of TNF binding to the cells in the presence of the TNF antagonist.

As another example, TNF antagonists may be analyzed for the ability to neutralize TNF activity in vitro in a bioassay using cells susceptible to the cytotoxic activity of TNF as target cells. In such an assay, target cells, cultured with TNF, are treated with varying amounts of TNF antagonist and subsequently are examined for cytolysis. TNF antagonist activity is assessed by a decrease in TNF-induced target cell cytolysis in the presence of the TNF antagonist.

### Treatment of NASH

In some aspects, the present invention provides methods of treating NASH, the methods generally involving administering to an individual in need thereof an effective amount of pirfenidone or a pirfenidone analog.

An effective amount of pirfenidone or a pirfenidone analog is an amount that is effective to reduce at least one sign or symptom or parameter associated with NASH by at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, or at least about 80% or more, when compared to the level or severity of the sign or symptom or parameter in an individual not treated with pirfenidone.

Symptoms of NASH include elevated alanine transaminase (ALT); elevated aspartate transaminase (AST); enlarged liver; increase in fat content of liver cells (as determined by histological examination of a liver biopsy sample). Thus, an effective amount of pirfenidone or a pirfenidone analog is an amount that is effective to reduce one or more of the level of ALT, the level of AST, liver mass, and fat content of liver cells by at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, or at least about 80% or more, when compared to the level of ALT, the level of AST, liver mass, or fat content of liver cells in an individual not treated with pirfenidone.

Treatment of NASH increases one or more liver functions. Liver functions include, but are not limited to, synthesis of proteins such as serum proteins (e.g., albumin, clotting factors, alkaline phosphatase, aminotransferases (e.g., alanine transaminase, aspartate transaminase), 5′-nucleosidase, γ-glutaminyltranspeptidase, etc.), synthesis of bilirubin, synthesis of cholesterol, and synthesis of bile acids; a liver metabolic function, including, but not limited to, carbohydrate metabolism, amino acid and ammonia metabolism, hormone metabolism, and lipid metabolism; detoxification of exogenous drugs; a hemodynamic function, including splanchnic and portal hemodynamics; and the like.

Whether a liver function is increased is readily ascertainable by those skilled in the art, using well-established tests of liver function. Thus, synthesis of markers of liver function such as albumin, alkaline phosphatase, alanine transaminase, aspartate transaminase, bilirubin, and the like, can be assessed by measuring the level of these markers in the serum, using standard immunological and enzymatic assays. Splanchnic circulation and portal hemodynamics can be measured by portal wedge pressure and/or resistance using standard methods. Metabolic functions can be measured by measuring the level of ammonia in the serum.

Whether serum proteins normally secreted by the liver are in the normal range can be determined by measuring the levels of such proteins, using standard immunological and enzymatic assays. Those skilled in the art know the normal ranges for such serum proteins. The following are non-limiting examples. The normal range of alanine transaminase is from about 7 to about 56 units per liter of serum. The normal range of aspartate transaminase is from about 5 to about 40 units per liter of serum. Bilirubin is measured using standard assays. Normal bilirubin levels are usually less than about 1.2 mg/dL. Serum albumin levels are measured using standard assays. Normal levels of serum albumin are in the range of from about 35 to about 55 g/L. Prolongation of prothrombin time is measured using standard assays. Normal prothrombin time is less than about 4 seconds longer than control.

In another embodiment, a therapeutically effective amount of pirfenidone or a pirfenidone analog is an amount that is effective to increase liver function by at least about 10%, at least about 20%, at least about 30%, at least about 40%, at least about 50%, at least about 60%, at least about 70%, at least about 80%, or more. For example, a therapeutically effective amount of pirfenidone or a pirfenidone analog is an amount that is effective to reduce an elevated level of a serum marker of liver function by at least about 10%, at least about 20%, at least about 30%, at least about 40%, at least about 50%, at least about 60%, at least about 70%, at least about 80%, or more, or to reduce the level of the serum marker of liver function to within a normal range. A therapeutically effective amount of pirfenidone or a pirfenidone analog is also an amount effective to increase a reduced level of a serum marker of liver function by at least about 10%, at least about 20%, at least about 30%, at least about 40%, at least about 50%, at least about 60%, at least about 70%, at least about 80%, or more, or to increase the level of the serum marker of liver function to within a normal range.

### Type II Diabetes Treatment

The invention provides a method of treating Type II diabetes, particularly advanced, end-stage Type II diabetes in which the afflicted individual is both insulin resistant and has reduced insulin production. The method generally involves administering an effective amount of pirfenidone or a pirfenidone analog and an effective amount of insulin.

An effective amount of pirfenidone or a pirfenidone analog, and an effective amount of insulin are amounts that, when administered to a patient having advanced Type II diabetes, result in a level of blood glucose that is closer to or within the normal range. Where the individual is hyperglycemic, effective amounts of pirfenidone or a pirfenidone analog and insulin are amounts effective to reduce blood glucose levels by at least about 10%, at least about 15%, at least about 20%, at least about 25%, at least about 30%, at least about 35%, or at least about 40% when compared with blood glucose levels in the patient not treated with pirfenidone and insulin combination therapy. In particular embodiments, effective amounts of pirfenidone or a pirfenidone analog and insulin are amounts effective to bring blood glucose levels within a normal range, e.g., to within 70 mg/dl to 110 mg/dl (e.g., a fasting blood glucose level normal range).

Whether blood glucose levels are within the normal range can be determined using standard assays, e.g., a fasting glucose test, or an oral glucose tolerance test.

The “gold standard” for diagnosing diabetes is an elevated blood sugar level after an overnight fast (not eating anything after midnight). A value above 140 mg/dl on at least two occasions typically means a person has diabetes. Normal (non-diabetic) people have fasting blood glucose levels that are generally between 70-110 mg/dl. When an individual has a fasting glucose equal to or greater than 110 mg/dl and less than 126 mg/dl, the individual is said to have impaired fasting glucose.

An oral glucose tolerance test is one that can be performed in a doctor's office or a lab. The person being tested starts the test in a fasting state (having no food or drink except water for at least 10 hours but not greater than 16 hours). An initial blood sugar is drawn and then the person is given a “glucola” bottle with a high amount of sugar in it (75 grams of glucose), (or 100 grams for pregnant women). The person then has his or her blood tested again 30 minutes, 1 hour, 2 hours and 3 hours after drinking the high glucose drink. A person has diabetes when oral glucose tolerance tests show that the blood glucose level at 2 hours is equal to or more than about 180 mg/dl to about 200 mg/dl.

### Insulin

As used herein, the term “insulin” includes regular or short-acting, intermediate-acting, and long-acting insulins; non-injectable or inhaled insulin; tissue selective insulin; D-chiroinositol; insulin analogs such as insulin molecules with minor differences in the natural amino acid sequence; small molecule mimics of insulin (insulin mimetics); endosome modulators. The term “insulin” includes any synthetic or recombinant insulin. Examples include, but are not limited to, insulin aspart (human insulin (28B-L-aspartic acid) or B28-Asp-insulin, also known as insulin X14, INA-X14, NOVORAPID, NOVOMIX, or NOVOLOG); insulin detemir (Human 29B-(N6-1-oxotetradecyl)-L-lysine)-(1A-21A), (1B-29B)-Insulin or NN 304); insulin lispro (“28B-L-lysine-29B-L-proline human insulin, or Lys(B28), Pro(B29) human insulin analog, also known as lys-pro insulin, LY 275585, HUMALOG, HUMALOG MIX 75/25, or HUMALOG MIX 50/50); insulin glargine (human (A21-glycine, B31-arginine, B32-arginine) insulin HOE 901, also known as LANTUS, OPTISULIN); Insulin Zinc Suspension, extended (Ultralente), also known as HUMULIN U or ULTRALENTE; Insulin Zinc suspension (Lente), a 70% crystalline and 30% amorphous insulin suspension, also known as LENTE ILETIN II, HUMULIN L, or NOVOLIN L; HUMULIN 50/50 (50% isophane insulin and 50% insulin injection); HUMULIN 70/30 (70% isophane insulin NPH and 30% insulin injection), also known as NOVOLIN 70/30, NOVOLIN 70/30 PenFill, NOVOLIN 70/30 Prefilled; insulin isophane suspension such as NPH ILETIN II, NOVOLIN N, NOVOLIN N PenFill, NOVOLIN N Prefilled, HUMULIN N; regular insulin injection such as ILETIN II Regular, NOVOLIN R, VELOSULIN BR, NOVOLIN R PenFill, NOVOLIN R Prefilled, HUMULIN R, or Regular U-500 (Concentrated).

### Pirfenidone and Analogs Thereof

Pirfenidone (5-methyl-1-phenyl-2-(1H)-pyridone) and specific pirfenidone analogs are disclosed for use in a subject treatment method.

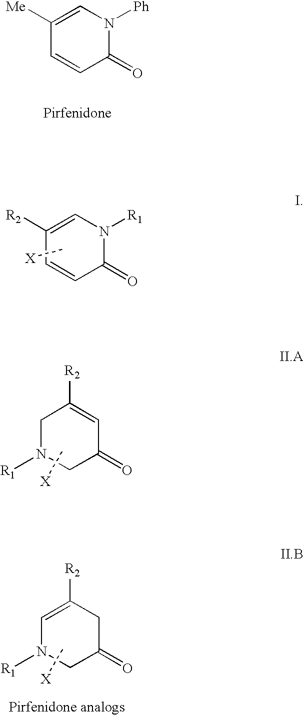

### Descriptions for Substituents R₁, R₂, X

R₁: carbocyclic (saturated and unsaturated), heterocyclic (saturated or unsaturated), alkyls (saturated and unsaturated). Examples include phenyl, benzyl, pyrimidyl, naphthyl, indolyl, pyrrolyl, furyl, thienyl, imidazolyl, cyclohexyl, piperidyl, pyrrolidyl, morpholinyl, cyclohexenyl, butadienyl, and the like.

R₁ can further include substitutions on the carbocyclic or heterocyclic moieties with substituents such as halogen, nitro, amino, hydroxyl, alkoxy, carboxyl, cyano, thio, alkyl, aryl, heteroalkyl, heteroaryl and combinations thereof, for example, 4-nitrophenyl, 3-chlorophenyl, 2,5-dinitrophenyl, 4-methoxyphenyl, 5-methyl-pyrrolyl, 2,5-dichlorocyclohexyl, guanidinyl-cyclohexenyl and the like.

R₂: alkyl, carbocylic, aryl, heterocyclic. Examples include: methyl, ethyl, propyl, isopropyl, phenyl, 4-nitrophenyl, thienyl and the like.

X: may be any number (from 1 to 3) of substituents on the carbocyclic or heterocyclic ring. The substituents can be the same or different. Substituents can include hydrogen, alkyl, heteroalkyl, aryl, heteroaryl, halo, nitro, carboxyl, hydroxyl, cyano, amino, thio, alkylamino, haloaryl and the like.

The substituents may be optionally further substituted with 1-3 substituents from the group consisting of alkyl, aryl, nitro, alkoxy, hydroxyl and halo groups. Examples include: methyl, 2,3-dimethyl, phenyl, p-tolyl, 4-chlorophenyl, 4-nitrophenyl, 2,5-dichlorophenyl, furyl, thienyl and the like.

Specific Examples include those shown in Table 1:

TABLE 1

IA
5-Methyl-1-(2′-pyridyl)-2-(1H) pyridine,
6-Methyl-1-phenyl-2-(1H) pyridone,
5-Methyl-3-phenyl-1-(2′-thienyl)-2-(1H) pyridone,
5-Methyl-1-(2′-naphthyl)-2-(1H) pyridone,
5-Methyl-1-p-tolyl-2-(1H) pyridone,
5-Methyl-1-(1′naphthyl)-2-(1H) pyridone,
5-Ethyl-1-phenyl-2-(1H) pyridone,
5-Methyl-1-(5′-quinolyl)-2-(1H) pyridone,
5-Methyl-1-(4′-quinolyl)-2-(1H) pyridone,
5-Methyl-1-(4′-pyridyl)-2-(1H) pyridone,
3-Methyl-1-phenyl-2-(1H) pyridone,
5-Methyl-1-(4′-methoxyphenyl)-2-(1H) pyridone,
1-Phenyl-2-(1H) pyridone,
1,3-Diphenyl-2-(1H) pyridone,
1,3-Diphenyl-5-methyl-2-(1H) pyridone,
5-Methyl-1-(3′-trifluoromethylphenyl)-2-(1H)-pyridone,
3-Ethyl-1-phenyl-2-(1H) pyridone,
5-Methyl-1-(3′-pyridyl)-2-(1H) pyridone,
5-Methyl-1-(3-nitrophenyl)-2-(1H) pyridone,
3-(4′-Chlorophenyl)-5-Methyl-1-phenyl-2-(1H) pyridone,
5-Methyl-1-(2′-Thienyl)-2-(1H) pyridone,
5-Methyl-1-(2′-thiazolyl)-2-(1H) pyridone,
3,6-Dimethyl-1-phenyl-2-(1H) pyridone,
1-(4′Chlorophenyl)-5-Methyl-2-(1H) pyridone,
1-(2′-Imidazolyl)-5-Methyl-2-(1H) pyridone,
1-(4′-Nitrophenyl)-2-(1H) pyridone,
1-(2′-Furyl)-5-Methyl-2-(1H) pyridone,
1-Phenyl-3-(4′-chlorophenyl)-2-(1H) pyridine.
IIB
6-Methyl-1-phenyl-3-(1H) pyridone,
5-Methyl-1-p-tolyl-3-(1H) pyridone,
5-Methyl-1-(2′-naphthyl)-3-(1H) pyridone,
5-Methyl-1-phenyl-3-(1H) pyridone,
5-Methyl-1-(5′-quinolyl)-3-(1H) pyridone,
5-Ethyl-1-phenyl-3-(1H) pyridone,
5-Methyl-1-(4′-methoxyphenyl)-3-(1H) pyridone,
4-Methyl-1-phenyl-3-(1H) pyridone,
5-Methyl-1-(3′-pyridyl)-3-(1H) pyridone,
5-Methyl-1-(2′-Thienyl)-3-(1H) pyridone,
5-Methyl-1-(2′-pyridyl)-3-(1H) pyridone,
5-Methyl-1-(2′-quinolyl)-3-(1H) pyridone,
1-Phenyl-3-(1H) pyridine,
1-(2′-Furyl)-5-methyl-3-(1H) pyridone,
1-(4′-Chlorophenyl)-5-methyl-3-(1H) pyridine.

U.S. Pat. Nos. 3,974,281; 3,839,346; 4,042,699; 4,052,509; 5,310,562; 5,518,729; 5,716,632; and 6,090,822 describe methods for the synthesis and formulation of pirfenidone and specific pirfenidone analogs in pharmaceutical compositions suitable for use in the methods of the present invention.

### Dosages, Formulations, and Routes of Administration

In carrying out a subject method, a therapeutic agent (also referred to herein as an “active agent”; e.g., pirfenidone, pirfenidone analog, a TNF-α antagonist, insulin, etc.) is administered to an individual in need thereof in a formulation (e.g., in separate formulations) with a pharmaceutically acceptable excipient(s). A wide variety of pharmaceutically acceptable excipients are known in the art and need not be discussed in detail herein. Pharmaceutically acceptable excipients have been amply described in a variety of publications, including, for example, A. Gennaro (2000) “Remington: The Science and Practice of Pharmacy”, 20th edition, Lippincott, Williams, & Wilkins; Pharmaceutical Dosage Forms and Drug Delivery Systems (1999) H. C. Ansel et al., eds 7^{th} ed., Lippincott, Williams, & Wilkins; and Handbook of Pharmaceutical Excipients (2000) A. H. Kibbe et al., eds., 3^{rd} ed. Amer. Pharmaceutical Assoc.

In the subject methods, an active agent (e.g., pirfenidone, pirfenidone analog, a TNF-α antagonist, insulin, etc.) may be administered to the host using any convenient means capable of resulting in the desired therapeutic effect. Thus, the agent can be incorporated into a variety of formulations for therapeutic administration. More particularly, the agents of the present invention can be formulated into pharmaceutical compositions by combination with appropriate, pharmaceutically acceptable carriers or diluents, and may be formulated into preparations in solid, semi-solid, liquid or gaseous forms, such as tablets, capsules, powders, granules, ointments, solutions, suppositories, injections, inhalants and aerosols.

As such, administration of an active agent (e.g., pirfenidone, pirfenidone analog, a TNF-α antagonist, insulin, etc.) can be achieved in various ways, including oral, buccal, rectal, parenteral, intraperitoneal, intradermal, intravenous, subcutaneous, intramuscular, intratumoral, transdermal, intratracheal, etc., administration. In some embodiments, two different routes of administration are used. For example, in some embodiments, a TNF-α antagonist is administered by a route such as intramuscular, subcutaneous, or intravenous, and pirfenidone or pirfenidone analog is administered orally.

Subcutaneous administration of a therapeutic agent is accomplished using standard methods and devices, e.g., needle and syringe, a subcutaneous injection port delivery system, and the like. See, e.g., U.S. Pat. Nos. 3,547,119; 4,755,173; 4,531,937; 4,311,137; and 6,017,328. A combination of a subcutaneous injection port and a device for administration of a therapeutic agent to a patient through the port is referred to herein as “a subcutaneous injection port delivery system.” In some embodiments, subcutaneous administration is achieved by a combination of devices, e.g., bolus delivery by needle and syringe, followed by delivery using a continuous delivery system.

In some embodiments, a therapeutic agent is delivered by a continuous delivery system. The term “continuous delivery system” is used interchangeably herein with “controlled delivery system” and encompasses continuous (e.g., controlled) delivery devices (e.g., pumps) in combination with catheters, injection devices, and the like, a wide variety of which are known in the art.

Mechanical or electromechanical infusion pumps can also be suitable for use with the present invention. Examples of such devices include those described in, for example, U.S. Pat. Nos. 4,692,147; 4,360,019; 4,487,603; 4,360,019; 4,725,852; 5,820,589; 5,643,207; 6,198,966; and the like. In general, drug delivery of an active agent (e.g., pirfenidone, pirfenidone analog, a TNF-α antagonist, insulin, etc.) can be accomplished using any of a variety of refillable, pump systems. Pumps provide consistent, controlled release over time. Typically, the agent is in a liquid formulation in a drug-impermeable reservoir, and is delivered in a continuous fashion to the individual.

In one embodiment, the drug delivery system is an at least partially implantable device. The implantable device can be implanted at any suitable implantation site using methods and devices well known in the art. An implantation site is a site within the body of a subject at which a drug delivery device is introduced and positioned. Implantation sites include, but are not necessarily limited to a subdermal, subcutaneous, intramuscular, or other suitable site within a subject's body. Subcutaneous implantation sites are generally preferred because of convenience in implantation and removal of the drug delivery device.

Drug release devices suitable for use in the invention may be based on any of a variety of modes of operation. For example, the drug release device can be based upon a diffusive system, a convective system, or an erodible system (e.g., an erosion-based system). For example, the drug release device can be an electrochemical pump, osmotic pump, an electroosmotic pump, a vapor pressure pump, or osmotic bursting matrix, e.g., where the drug is incorporated into a polymer and the polymer provides for release of drug formulation concomitant with degradation of a drug-impregnated polymeric material (e.g., a biodegradable, drug-impregnated polymeric material). In other embodiments, the drug release device is based upon an electrodiffusion system, an electrolytic pump, an effervescent pump, a piezoelectric pump, a hydrolytic system, etc.

Drug release devices based upon a mechanical or electromechanical infusion pump can also be suitable for use with the present invention. Examples of such devices include those described in, for example, U.S. Pat. Nos. 4,692,147; 4,360,019; 4,487,603; 4,360,019; 4,725,852, and the like. In general, delivery of an active agent (e.g., pirfenidone, pirfenidone analog, a TNF-α antagonist, insulin, etc.) can be accomplished using any of a variety of refillable, non-exchangeable pump systems. Pumps and other convective systems are generally preferred due to their generally more consistent, controlled release over time. Osmotic pumps are particularly preferred due to their combined advantages of more consistent controlled release and relatively small size (see, e.g., PCT published application no. WO 97/27840 and U.S. Pat. Nos. 5,985,305 and 5,728,396)). Exemplary osmotically-driven devices suitable for use in the invention include, but are not necessarily limited to, those described in U.S. Pat. Nos. 3,760,984; 3,845,770; 3,916,899; 3,923,426; 3,987,790; 3,995,631; 3,916,899; 4,016,880; 4,036,228; 4,111,202; 4,111,203; 4,203,440; 4,203,442; 4,210,139; 4,327,725; 4,627,850; 4,865,845; 5,057,318; 5,059,423; 5,112,614; 5,137,727; 5,234,692; 5,234,693; 5,728,396; and the like.

In some embodiments, the drug delivery device is an implantable device. The drug delivery device can be implanted at any suitable implantation site using methods and devices well known in the art. As noted infra, an implantation site is a site within the body of a subject at which a drug delivery device is introduced and positioned. Implantation sites include, but are not necessarily limited to a subdermal, subcutaneous, intramuscular, or other suitable site within a subject's body.

In some embodiments, a therapeutic agent (e.g., pirfenidone, pirfenidone analog, a TNF-α antagonist, insulin, etc.) is delivered using an implantable drug delivery system, e.g., a system that is programmable to provide for administration of the therapeutic agent. Exemplary programmable, implantable systems include implantable infusion pumps. Exemplary implantable infusion pumps, or devices useful in connection with such pumps, are described in, for example, U.S. Pat. Nos. 4,350,155; 5,443,450; 5,814,019; 5,976,109; 6,017,328; 6,171,276; 6,241,704; 6,464,687; 6,475,180; and 6,512,954. A further exemplary device that can be adapted for the present invention is the Synchromed infusion pump (Medtronic).

In pharmaceutical dosage forms, the agents may be administered in the form of their pharmaceutically acceptable salts, or they may also be used alone or in appropriate association, as well as in combination, with other pharmaceutically active compounds. The following methods and excipients are merely exemplary and are in no way limiting.

For oral preparations, an active agent (e.g., pirfenidone, pirfenidone analog, a TNF-α antagonist, insulin, etc.) can be used alone or in combination with appropriate additives to make tablets, powders, granules or capsules, for example, with conventional additives, such as lactose, mannitol, corn starch or potato starch; with binders, such as crystalline cellulose, cellulose derivatives, acacia, corn starch or gelatins; with disintegrators, such as corn starch, potato starch or sodium carboxymethylcellulose; with lubricants, such as talc or magnesium stearate; and if desired, with diluents, buffering agents, moistening agents, preservatives and flavoring agents.

The agents can be formulated into preparations for injection by dissolving, suspending or emulsifying them in an aqueous or nonaqueous solvent, such as vegetable or other similar oils, synthetic aliphatic acid glycerides, esters of higher aliphatic acids or propylene glycol; and if desired, with conventional additives such as solubilizers, isotonic agents, suspending agents, emulsifying agents, stabilizers and preservatives.

Furthermore, the agents can be made into suppositories by mixing with a variety of bases such as emulsifying bases or water-soluble bases. An active agent (e.g., pirfenidone, pirfenidone analog, a TNF-α antagonist, insulin, etc.) can be administered rectally via a suppository. The suppository can include vehicles such as cocoa butter, carbowaxes and polyethylene glycols, which melt at body temperature, yet are solidified at room temperature.

Unit dosage forms for oral or rectal administration such as syrups, elixirs, and suspensions may be provided wherein each dosage unit, for example, teaspoonful, tablespoonful, tablet or suppository, contains a predetermined amount of one or more active agents. Similarly, unit dosage forms for injection or intravenous administration may comprise an active agent(s) in a composition as a solution in sterile water, normal saline or another pharmaceutically acceptable carrier.

The term “unit dosage form,” as used herein, refers to physically discrete units suitable as unitary dosages for human and animal subjects, each unit containing a predetermined quantity of an active agent, calculated in an amount sufficient to produce the desired effect in association with a pharmaceutically acceptable diluent, carrier or vehicle. The specifications for an active agent depend on the particular compound employed and the effect to be achieved, and the pharmacodynamics associated with each compound in the host.

The pharmaceutically acceptable excipients, such as vehicles, adjuvants, carriers or diluents, are readily available to the public. Moreover, pharmaceutically acceptable auxiliary substances, such as pH adjusting and buffering agents, tonicity adjusting agents, stabilizers, wetting agents and the like, are readily available to the public.

Where the administered therapeutic agent is a polypeptide (e.g., an anti-TNF-α monoclonal antibody, a soluble TNF receptor, etc.) a polynucleotide encoding the polypeptide therapeutic agent may be introduced into tissues or host cells by any number of routes, including viral infection, microinjection, or fusion of vesicles. Jet injection may also be used for intramuscular administration, as described by Furth et al. (1992), Anal Biochem 205:365-368. The DNA may be coated onto gold microparticles, and delivered intradermally by a particle bombardment device, or “gene gun” as described in the literature (see, for example, Tang et al. (1992), Nature 356:152-154), where gold microprojectiles are coated with the therapeutic DNA, then bombarded into skin cells.

In general, effective dosages of pirfenidone or specific pirfenidone analogs can range from about 0.5 mg/kg/day to about 200 mg/kg/day, or at a fixed dosage of about 400 mg to about 3600 mg per day, or about 50 mg to about 5,000 mg per day, or about 100 mg to about 1,000 mg per day, administered orally, optionally in two or more divided doses per day. Other doses and formulations of pirfenidone and pirfenidone analogs suitable for use in a subject method for the treatment of inflammatory diseases, fibrotic disorders, TNF-mediated diseases, cancer, and cytokine growth factor-mediated disorders are described in U.S. Pat. Nos. 3,974,281; 3,839,346; 4,042,699; 4,052,509; 5,310,562; 5,518,729; 5,716,632; and 6,090,822.

Those of skill in the art will readily appreciate that dose levels of pirfenidone or pirfenidone analog can vary as a function of the specific compound, the severity of the symptoms and the susceptibility of the subject to side effects. Preferred dosages for a given compound are readily determinable by those of skill in the art by a variety of means.

Pirfenidone (or a pirfenidone analog) can be administered daily, twice a day, or three times a day, or in divided daily doses ranging from 2 to 5 times daily over a period of time ranging from about one day to about one week, from about two weeks to about four weeks, from about one month to about two months, from about two months to about four months, from about four months to about six months, from about six months to about eight months, from about eight months to about 1 year, from about 1 year to about 2 years, or from about 2 years to about 4 years, or more.

In some embodiments, in which two therapeutic agents are administered, the second therapeutic agent is administered during the entire course of treatment with the first therapeutic agent. In other embodiments, the second therapeutic agent is administered for a period of time that is overlapping with that of the first therapeutic agent, e.g., the second therapeutic agent treatment can begin before the first therapeutic agent treatment begins and end before the first therapeutic agent treatment ends; the second therapeutic agent treatment can begin after the first therapeutic agent treatment begins and end after the first therapeutic agent treatment ends; the second therapeutic agent treatment can begin after the first therapeutic agent treatment begins and end before the first therapeutic agent treatment ends; or the second therapeutic agent treatment can begin before the first therapeutic agent treatment begins and end after the first therapeutic agent treatment ends.

### Pirfenidone in Combination Therapy with a TNF-α Antagonist

In some embodiments, a TNF-α antagonist is administered in combination therapy with pirdenidone or a pirfenidone analog for the treatment of a TNF-α-mediated disorder. The methods generally involve administering to an individual in need thereof an effective amount of a TNF-α antagonist and an effective amount of pirfenidone or pirfenidone analog.

In general, effective dosages of pirfenidone or specific pirfenidone analogs can range from about 0.5 mg/kg/day to about 200 mg/kg/day, or at a fixed dosage of about 400 mg to about 3600 mg per day, or about 50 mg to about 5,000 mg per day, or about 100 mg to about 1,000 mg per day, administered orally, optionally in two or more divided doses per day. Other doses and formulations of pirfenidone and pirfenidone analogs suitable for use in a subject method for the treatment of inflammatory disorders, fibrotic disorders, TNF-mediated disorders, cancer, and cytokine growth factor-mediated disorders, are described in U.S. Pat. Nos. 3,974,281; 3,839,346; 4,042,699; 4,052,509; 5,310,562; 5,518,729; 5,716,632; and 6,090,822.

Those of skill in the art will readily appreciate that dose levels of pirfenidone or pirfenidone analog can vary as a function of the specific compound, the severity of the symptoms and the susceptibility of the subject to side effects. Preferred dosages for a given compound are readily determinable by those of skill in the art by a variety of means.

Pirfenidone (or a pirfenidone analog) can be administered daily, twice a day, or three times a day, or in divided daily doses ranging from 2 to 5 times daily over a period of time ranging from about one day to about one week, from about two weeks to about four weeks, from about one month to about two months, from about two months to about four months, from about four months to about six months, from about six months to about eight months, from about eight months to about 1 year, from about 1 year to about 2 years, or from about 2 years to about 4 years, or more.

Effective dosages of a TNF-α antagonist range from 0.1 μg to 40 mg per dose, e.g., from about 0.1 μg to about 0.5 μg per dose, from about 0.5 μg to about 1.0 μg per dose, from about 1.0 μg per dose to about 5.0 μg per dose, from about 5.0 μg to about 10 μg per dose, from about 10 μg to about 20 μg per dose, from about 20 μg per dose to about 30 μg per dose, from about 30 μg per dose to about 40 μg per dose, from about 40 μg per dose to about 50 μg per dose, from about 50 μg per dose to about 60 μg per dose, from about 60 μg per dose to about 70 μg per dose, from about 70 μg to about 80 μg per dose, from about, 80 μg per dose to about 100 μg per dose, from about 100 μg to about 150 μg per dose, from about 150 μg to about 200 μg per dose, from about 200 μg per dose to about 250 μg per dose, from about 250 μg to about 300 μg per dose, from about 300 μg to about 400 μg per dose, from about 400 μg to about 500 μg per dose, from about 500 μg to about 600 μg per dose, from about 600 μg to about 700 μg per dose, from about 700 μg to about 800 μg per dose, from about 800 μg to about 900 μg per dose, from about 900 μg to about 1000 μg per dose, from about 1 mg to about 10 mg per dose, from about 10 mg to about 15 mg per dose, from about 15 mg to about 20 mg per dose, from about 20 mg to about 25 mg per dose, from about 25 mg to about 30 mg per dose, from about 30 mg to about 35 mg per dose, or from about 35 mg to about 40 mg per dose.

In some embodiments, effective dosages of a TNF-α antagonist are expressed as mg/kg body weight. In these embodiments, effective dosages of a TNF-α antagonist are from about 0.1 mg/kg body weight to about 10 mg/kg body weight, e.g., from about 0.1 mg/kg body weight to about 0.5 mg/kg body weight, from about 0.5 mg/kg body weight to about 1.0 mg/kg body weight, from about 1.0 mg/kg body weight to about 2.5 mg/kg body weight, from about 2.5 mg/kg body weight to about 5.0 mg/kg body weight, from about 5.0 mg/kg body weight to about 7.5 mg/kg body weight, or from about 7.5 mg/kg body weight to about 10 mg/kg body weight.

In many embodiments, a TNF-α antagonist is administered for a period of about 1 day to about 7 days, or about 1 week to about 2 weeks, or about 2 weeks to about 3 weeks, or about 3 weeks to about 4 weeks, or about 1 month to about 2 months, or about 3 months to about 4 months, or about 4 months to about 6 months, or about 6 months to about 8 months, or about 8 months to about 12 months, or at least one year, and may be administered over longer periods of time. The TNF-α antagonist can be administered tid, bid, qd, qod, biw, tiw, qw, qow, three times per month, once monthly, substantially continuously, or continuously.

In many embodiments, multiple doses of a TNF-α antagonist are administered. For example, a TNF-α antagonist is administered once per month, twice per month, three times per month, every other week (qow), once per week (qw), twice per week (biw), three times per week (tiw), four times per week, five times per week, six times per week, every other day (qod), daily (qd), twice a day (bid), or three times a day (tid), substantially continuously, or continuously, over a period of time ranging from about one day to about one week, from about two weeks to about four weeks, from about one month to about two months, from about two months to about four months, from about four months to about six months, from about six months to about eight months, from about eight months to about 1 year, from about 1 year to about 2 years, or from about 2 years to about 4 years, or more.

A TNF-α antagonist and pirfenidone (or pirfenidone analog) are generally administered in separate formulations. A TNF-α antagonist and pirfenidone (or pirfenidone analog) may be administered substantially simultaneously, or within about 30 minutes, about 1 hour, about 2 hours, about 4 hours, about 8 hours, about 16 hours, about 24 hours, about 36 hours, about 72 hours, about 4 days, about 7 days, or about 2 weeks of one another.

In one embodiment, the invention provides a method using an effective amount of a TNF-α antagonist and pirfenidone or a specific pirfenidone analog in the treatment of a TNF-α-mediated disorder in a patient, comprising administering to the patient a dosage of a TNF-α antagonist containing an amount of from about 0.1 μg to about 40 mg per dose of a TNF-α antagonist, subcutaneously qd, qod, tiw, or biw, or per day substantially continuously or continuously, in combination with a dosage of pirfenidone or a specific pirfenidone analog containing an amount of about 100 mg to about 1,000 mg of drug per dose of pirfenidone or a specific pirfenidone analog orally qd, optionally in two or more divided doses per day, for the desired treatment duration.

In one embodiment, the invention provides a method using an effective amount of a TNF-α antagonist and pirfenidone or a specific pirfenidone analog in the treatment of a TNF-α-mediated disorder in a patient, comprising administering to the patient a dosage of a TNF-α antagonist containing an amount of from about 0.1 μg to about 40 mg per dose of a TNF-α antagonist, subcutaneously qd, qod, tiw, or biw, or per day substantially continuously or continuously, in combination with a dosage of pirfenidone or a specific pirfenidone analog containing an amount of about 50 mg to about 5,000 mg of drug per dose of pirfenidone or a specific pirfenidone analog orally qd, optionally in two or more divided doses per day, for the desired treatment duration.

In one embodiment, the invention provides a method using an effective amount of a TNF-α antagonist and pirfenidone or a specific pirfenidone analog in the treatment of a TNF-α-mediated disorder in a patient, comprising administering to the patient a dosage of a TNF-α antagonist containing an amount of from about 0.1 μg to about 40 mg per dose of a TNF-α antagonist, subcutaneously qd, qod, tiw, or biw, or per day substantially continuously or continuously, in combination with a dosage of pirfenidone or a specific pirfenidone analog containing an amount of about 500 mg of drug per dose of pirfenidone or a specific pirfenidone analog orally qd, optionally in two or more divided doses per day, for the desired treatment duration.

In one embodiment, the invention provides a method using an effective amount of ENBREL® and pirfenidone or a specific pirfenidone analog in the treatment of a TNF-α-mediated disorder in a patient, comprising administering to the patient a dosage ENBREL® containing an amount of from about 0.1 μg to about 23 mg per dose, from about 0.1 μg to about 1 μg, from about 1 μg to about 10 μg, from about 10 μg to about 100 μg, from about 100 μg to about 1 mg, from about 1 mg to about 5 mg, from about 5 mg to about 10 mg, from about 10 mg to about 15 mg, from about 15 mg to about 20 mg, or from about 20 mg to about 23 mg of ENBREL®, subcutaneously qd, qod, tiw, biw, qw, qow, three times per month, once monthly, or once every other month, or per day substantially continuously or continuously, in combination with a dosage of pirfenidone or a specific pirfenidone analog containing an amount of about 100 mg to about 1,000 mg of drug per dose of pirfenidone or a specific pirfenidone analog orally qd, optionally in two or more divided doses per day, for the desired treatment duration.

In one embodiment, the invention provides a method using an effective amount of ENBREL® and pirfenidone or a specific pirfenidone analog in the treatment of a TNF-α-mediated disorder in a patient, comprising administering to the patient a dosage ENBRELS containing an amount of from about 0.1 μg to about 23 mg per dose, from about 0.1 μg to about 1 μg, from about 1 μg to about 10 μg, from about 10 μg to about 100 μg, from about 100 μg to about 1 mg, from about 1 mg to about 5 mg, from about 5 mg to about 10 mg, from about 10 mg to about 15 mg, from about 15 mg to about 20 mg, or from about 20 mg to about 23 mg of ENBREL®, subcutaneously qd, qod, tiw, biw, qw, qow, three times per month, once monthly, or once every other month, or per day substantially continuously or continuously, in combination with a dosage of pirfenidone or a specific pirfenidone analog containing an amount of about 50 mg to about 5,000 mg of drug per dose of pirfenidone or a specific pirfenidone analog orally qd, optionally in two or more divided doses per day, for the desired treatment duration.

In one embodiment, the invention provides a method using an effective amount of ENBREL® and pirfenidone or a specific pirfenidone analog in the treatment of a TNF-α-mediated disorder in a patient, comprising administering to the patient a dosage ENBREL® containing an amount of from about 0.1 μg to about 23 mg per dose, from about 0.1 μg to about 1 μg, from about 1 μg to about 10 μg, from about 10 μg to about 100 μg, from about 100 μg to about 1 mg, from about 1 mg to about 5 mg, from about 5 mg to about 10 mg, from about 10 mg to about 15 mg, from about 15 mg to about 20 mg, or from about 20 mg to about 23 mg of ENBREL®, subcutaneously qd, qod, tiw, biw, qw, qow, three times per month, once monthly, or once every other month, or per day substantially continuously or continuously, in combination with a dosage of pirfenidone or a specific pirfenidone analog containing an amount of about 500 mg of drug per dose of pirfenidone or a specific pirfenidone analog orally qd, optionally in two or more divided doses per day, for the desired treatment duration.

In one embodiment, the invention provides a method using an effective amount of REMICADE® and pirfenidone or a specific pirfenidone analog in the treatment of a TNF-α-mediated disorder in a patient, comprising administering to the patient a dosage of REMICADE® containing an amount of from about 0.1 mg/kg to about 4.5 mg/kg, from about 0.1 mg/kg to about 0.5 mg/kg, from about 0.5 mg/kg to about 1.0 mg/kg, from about 1.0 mg/kg to about 1.5 mg/kg, from about 1.5 mg/kg to about 2.0 mg/kg, from about 2.0 mg/kg to about 2.5 mg/kg, from about 2.5 mg/kg to about 3.0 mg/kg, from about 3.0 mg/kg to about 3.5 mg/kg, from about 3.5 mg/kg to about 4.0 mg/kg, or from about 4.0 mg/kg to about 4.5 mg/kg per dose of REMICADE®, intravenously qd, qod, tiw, biw, qw, qow, three times per month, once monthly, or once every other month, or per day substantially continuously or continuously, in combination with a dosage of pirfenidone or a specific pirfenidone analog containing an amount of about 100 mg to about 1,000 mg of drug per dose of pirfenidone or a specific pirfenidone analog orally qd, optionally in two or more divided doses per day, for the desired treatment duration.

In one embodiment, the invention provides a method using an effective amount of REMICADE® and pirfenidone or a specific pirfenidone analog in the treatment of a TNF-α-mediated disorder in a patient, comprising administering to the patient a dosage of REMICADE® containing an amount of from about 0.1 mg/kg to about 4.5 mg/kg, from about 0.1 mg/kg to about 0.5 mg/kg, from about 0.5 mg/kg to about 1.0 mg/kg, from about 1.0 mg/kg to about 1.5 mg/kg, from about 1.5 mg/kg to about 2.0 mg/kg, from about 2.0 mg/kg to about 2.5 mg/kg, from about 2.5 mg/kg to about 3.0 mg/kg, from about 3.0 mg/kg to about 3.5 mg/kg, from about 3.5 mg/kg to about 4.0 mg/kg, or from about 4.0 mg/kg to about 4.5 mg/kg per dose of REMICADE®, intravenously qd, qod, tiw, biw, qw, qow, three times per month, once monthly, or once every other month, or per day substantially continuously or continuously, in combination with a dosage of pirfenidone or a specific pirfenidone analog containing an amount of about 50 mg to about 5,000 mg of drug per dose of pirfenidone or a specific pirfenidone analog orally qd, optionally in two or more divided doses per day, for the desired treatment duration.

In one embodiment, the invention provides a method using an effective amount of REMICADE® and pirfenidone or a specific pirfenidone analog in the treatment of a TNF-α-mediated disorder in a patient, comprising administering to the patient a dosage of REMICADE® containing an amount of from about 0.1 mg/kg to about 4.5 mg/kg, from about 0.1 mg/kg to about 0.5 mg/kg, from about 0.5 mg/kg to about 1.0 mg/kg, from about 1.0 mg/kg to about 1.5 mg/kg, from about 1.5 mg/kg to about 2.0 mg/kg, from about 2.0 mg/kg to about 2.5 mg/kg, from about 2.5 mg/kg to about 3.0 mg/kg, from about 3.0 mg/kg to about 3.5 mg/kg, from about 3.5 mg/kg to about 4.0 mg/kg, or from about 4.0 mg/kg to about 4.5 mg/kg per dose of REMICADE®, intravenously qd, qod, tiw, biw, qw, qow, three times per month, once monthly, or once every other month, or per day substantially continuously or continuously, in combination with a dosage of pirfenidone or a specific pirfenidone analog containing an amount of about 500 mg of drug per dose of pirfenidone or a specific pirfenidone analog orally qd, optionally in two or more divided doses per day, for the desired treatment duration.

In one embodiment, the invention provides a method using an effective amount of HUMIRA™ and pirfenidone or a specific pirfenidone analog in the treatment of a TNF-α-mediated disorder in a patient, comprising administering to the patient a dosage of HUMIRA™ containing an amount of from about 0.1 μg to about 35 mg, from about 0.1 μg to about 1 μg, from about 1 μg to about 10 μg, from about 10 μg to about 100 μg, from about 100 μg to about 1 mg, from about 1 mg to about 5 mg, from about 5 mg to about 10 mg, from about 10 mg to about 15 mg, from about 15 mg to about 20 mg, from about 20 mg to about 25 mg, from about 25 mg to about 30 mg, or from about 30 mg to about 35 mg per dose of a HUMIRA™, subcutaneously qd, qod, tiw, biw, qw, qow, three times per month, once monthly, or once every other month, or per day substantially continuously or continuously, in combination with a dosage of pirfenidone or a specific pirfenidone analog containing an amount of about 100 mg to about 1,000 mg of drug per dose of pirfenidone or a specific pirfenidone analog orally qd, optionally in two or more divided doses per day, for the desired treatment duration.

In one embodiment, the invention provides a method using an effective amount of HUMIRA™ and pirfenidone or a specific pirfenidone analog in the treatment of a TNF-α-mediated disorder in a patient, comprising administering to the patient a dosage of HUMIRA™ containing an amount of from about 0.1 μg to about 35 mg, from about 0.1 μg to about 1 μg, from about 1 μg to about 10 μg, from about 10 μg to about 100 μg, from about 100 μg to about 1 mg, from about 1 mg to about 5 mg, from about 5 mg to about 10 mg, from about 10 mg to about 15 mg, from about 15 mg to about 20 mg, from about 20 mg to about 25 mg, from about 25 mg to about 30 mg, or from about 30 mg to about 35 mg per dose of a HUMIRA™, subcutaneously qd, qod, tiw, biw, qw, qow, three times per month, once monthly, or once every other month, or per day substantially continuously or continuously, in combination with a dosage of pirfenidone or a specific pirfenidone analog containing an amount of about 50 mg to about 5,000 mg of drug per dose of pirfenidone or a specific pirfenidone analog orally qd, optionally in two or more divided doses per day, for the desired treatment duration.

In one embodiment, the invention provides a method using an effective amount of HUMIRA™ and pirfenidone or a specific pirfenidone analog in the treatment of a TNF-α-mediated disorder in a patient, comprising administering to the patient a dosage of HUMIRA™ containing an amount of from about 0.1 μg to about 35 mg, from about 0.1 μg to about 1 μg, from about 1 μg to about 10 μg, from about 10 μg to about 100 μg, from about 100 μg to about 1 mg, from about 1 mg to about 5 mg, from about 5 mg to about 10 mg, from about 10 mg to about 15 mg, from about 15 mg to about 20 mg, from about 20 mg to about 25 mg, from about 25 mg to about 30 mg, or from about 30 mg to about 35 mg per dose of a HUMIRA™, subcutaneously qd, qod, tiw, biw, qw, qow, three times per month, once monthly, or once every other month, or per day substantially continuously or continuously, in combination with a dosage of pirfenidone or a specific pirfenidone analog containing an amount of about 500 mg of drug per dose of pirfenidone or a specific pirfenidone analog orally qd, optionally in two or more divided doses per day, for the desired treatment duration.

### Pirfenidone in Combination with Insulin

In some embodiments, pirdenidone or a pirfenidone analog is administered in combination therapy with insulin for the treatment of Type II diabetes. The methods generally involve administering to an individual in need thereof an effective amount of pirfenidone or pirfenidone analog and an effective amount of insulin.

In general, effective dosages of pirfenidone or specific pirfenidone analogs can range from about 0.5 mg/kg/day to about 200 mg/kg/day, or at a fixed dosage of about 400 mg to about 3600 mg per day, or about 50 mg to about 5,000 mg per day, or about 100 mg to about 1,000 mg per day, administered orally, optionally in two or more divided doses per day. Other doses and formulations of pirfenidone and pirfenidone analogs suitable for use in a subject method for the treatment of inflammatory disorders, fibrotic disorders, TNF-mediated disorders, cancer, and cytokine growth factor-mediated disorders, are described in U.S. Pat. Nos. 3,974,281; 3,839,346; 4,042,699; 4,052,509; 5,310,562; 5,518,729; 5,716,632; and 6,090,822.

Those of skill in the art will readily appreciate that dose levels of pirfenidone or pirfenidone analog can vary as a function of the specific compound, the severity of the symptoms and the susceptibility of the subject to side effects. Preferred dosages for a given compound are readily determinable by those of skill in the art by a variety of means.

Pirfenidone (or a pirfenidone analog) can be administered daily, twice a day, or three times a day, or in divided daily doses ranging from 2 to 5 times daily over a period of time ranging from about one day to about one week, from about two weeks to about four weeks, from about one month to about two months, from about two months to about four months, from about four months to about six months, from about six months to about eight months, from about eight months to about 1 year, from about 1 year to about 2 years, or from about 2 years to about 4 years, or more.

In general, effective dosages of insulin can range from about 0.5 Unit to about 50 Units per dose, e.g., from about 0.1 Unit to about 1 Unit, from about 1 Unit to about 5 Units (e.g., a dose of 1 Unit, 2 Units, 3 Units, 4 Units, or 5 Units), from about 5 Units to about 10 Units (e.g., a dose of 5 Units, 6 Units, 7 Units, 8 Units, 9 Units, or 10 Units), from about 10 Units to about 12 Units, from about 12 Units to about 15 Units, from about 15 Units to about 20 Units, from about 20 Units to about 25 Units, from about 25 Units to about 30 Units, from about 30 Units to about 35 Units, from about 35 Units to about 40 Units, from about 40 Units to about 45 Units, or about 45 Units to about 50 Units per dose. In many embodiments, insulin is administered intramuscularly or subcutaneously, by bolus injection or by continuous infusion with a pump.

Those of skill in the art will readily appreciate that dose levels of insulin can vary as a function of the specific insulin being used, the level of blood glucose, the anticipated timing of the next meal, etc. Preferred dosages for a given insulin are readily determinable by those of skill in the art by a variety of means. In some embodiments, the actual amount of insulin administered will depend upon the level of blood glucose. Thus, for example, if the blood glucose level is 101-150 mg/dl, then the regular insulin dose is administered; if the blood glucose level is 151-200 mg/dl, then the regular insulin dose plus 1 Unit is administered; if the blood glucose level is 201-250 mg/dl, then the regular insulin dose plus 2 Units is administered; if the blood glucose level is 251-300 mg/dl, then the regular insulin dose plus 3 Units is administered; if the blood glucose level is 301-350 mg/dl, then the regular insulin dose plus 4 Units is administered; and if the blood glucose level is greater than 350 mg/dl, then the regular insulin dose plus 5 Units is administered. As another example, if the blood glucose level is 240 mg/dl or greater, the regular dose plus ⅓ the regular dose of insulin is administered.

In many embodiments, insulin is administered for a period of about 1 day to about 7 days, or about 1 week to about 2 weeks, or about 2 weeks to about 3 weeks, or about 3 weeks to about 4 weeks, or about 1 month to about 2 months, or about 3 months to about 4 months, or about 4 months to about 6 months, or about 6 months to about 8 months, or about 8 months to about 12 months, or at least one year, and may be administered over longer periods of time. The insulin can be administered tid, bid, qd, qod, biw, tiw, qw, qow, three times per month, once monthly, substantially continuously, or continuously.

In many embodiments, multiple doses of insulin are administered. For example, insulin is administered once per month, twice per month, three times per month, every other week (qow), once per week (qw), twice per week (biw), three times per week (tiw), four times per week, five times per week, six times per week, every other day (qod), daily (qd), twice a day (qid), or three times a day (tid), substantially continuously, or continuously, over a period of time ranging from about one day to about one week, from about two weeks to about four weeks, from about one month to about two months, from about two months to about four months, from about four months to about six months, from about six months to about eight months, from about eight months to about 1 year, from about 1 year to about 2 years, or from about 2 years to about 4 years, or more. In many embodiments, insulin is administered substantially continuously, using, e.g., an osmotic pump.

In many embodiments, the frequency of administration of insulin depends upon the level of blood glucose. Blood glucose is monitored at regular intervals, or at specific times, e.g., after meals, etc. Depending on the blood glucose level, insulin may be administered more frequently or less frequently, as necessary to maintain blood glucose levels at or near a normal range. Methods of monitoring and detecting blood glucose are well known in the art.

Insulin and pirfenidone (or pirfenidone analog) are generally administered in separate formulations. Insulin and pirfenidone (or pirfenidone analog) may be administered substantially simultaneously, or within about 30 minutes, about 1 hour, about 2 hours, about 4 hours, about 8 hours, about 16 hours, about 24 hours, about 36 hours, about 72 hours, about 4 days, about 7 days, or about 2 weeks of one another.

In one embodiment, the invention provides a method using an effective amount of insulin and pirfenidone or a specific pirfenidone analog in the treatment of Type II diabetes in a patient, comprising administering to the patient a dosage of insulin containing an amount of from about 0.5 Unit to about 50 Units per dose of insulin, intramuscularly or subcutaneously qd, qod, tiw, or biw, or per day substantially continuously or continuously, in combination with a dosage of pirfenidone or a specific pirfenidone analog containing an amount of about 100 mg to about 1,000 mg of drug per dose of pirfenidone or a specific pirfenidone analog orally qd, optionally in two or more divided doses per day, for the desired treatment duration.

In one embodiment, the invention provides a method using an effective amount of insulin and pirfenidone or a specific pirfenidone analog in the treatment of Type II diabetes in a patient, comprising administering to the patient a dosage of insulin containing an amount of from about 0.5 Unit to about 50 Units per dose of insulin, intramuscularly or subcutaneously qd, qod, tiw, or biw, or per day substantially continuously or continuously, in combination with a dosage of pirfenidone or a specific pirfenidone analog containing an amount of about 50 mg to about 5,000 mg of drug per dose of pirfenidone or a specific pirfenidone analog orally qd, optionally in two or more divided doses per day, for the desired treatment duration.

In one embodiment, the invention provides a method using an effective amount of insulin and pirfenidone or a specific pirfenidone analog in the treatment of Type II diabetes in a patient, comprising administering to the patient a dosage of insulin containing an amount of from about 0.5 Unit to about 50 Units per dose of insulin, intramuscularly or subcutaneously qd, qod, tiw, or biw, or per day substantially continuously or continuously, in combination with a dosage of pirfenidone or a specific pirfenidone analog containing an amount of about 500 mg of drug per dose of pirfenidone or a specific pirfenidone analog orally qd, optionally in two or more divided doses per day, for the desired treatment duration.

In one embodiment, the invention provides a method using an effective amount of insulin and pirfenidone or a specific pirfenidone analog in the treatment of Type II diabetes in a patient, comprising administering to the patient a dosage of insulin containing an amount of from about 0.5 Unit to about 50 Units per dose of insulin aspart, intramuscularly or subcutaneously qd, qod, tiw, or biw, or per day substantially continuously or continuously, in combination with a dosage of pirfenidone or a specific pirfenidone analog containing an amount of about 100 mg to about 1,000 mg, or from about 500 mg to about 5,000 of drug per dose of pirfenidone or a specific pirfenidone analog orally qd, optionally in two or more divided doses per day, for the desired treatment duration.

In one embodiment, the invention provides a method using an effective amount of insulin and pirfenidone or a specific pirfenidone analog in the treatment of Type II diabetes in a patient, comprising administering to the patient a dosage of insulin containing an amount of from about 0.5 Unit to about 50 Units per dose of detemir, intramuscularly or subcutaneously qd, qod, tiw, or biw, or per day substantially continuously or continuously, in combination with a dosage of pirfenidone or a specific pirfenidone analog containing an amount of about 100 mg to about 1,000 mg, or from about 500 mg to about 5,000 of drug per dose of pirfenidone or a specific pirfenidone analog orally qd, optionally in two or more divided doses per day, for the desired treatment duration.

In one embodiment, the invention provides a method using an effective amount of insulin and pirfenidone or a specific pirfenidone analog in the treatment of Type II diabetes in a patient, comprising administering to the patient a dosage of insulin containing an amount of from about 0.5 Unit to about 50 Units per dose of insulin lispro, intramuscularly or subcutaneously qd, qod, tiw, or biw, or per day substantially continuously or continuously, in combination with a dosage of pirfenidone or a specific pirfenidone analog containing an amount of about 100 mg to about 1,000 mg, or from about 500 mg to about 5,000 of drug per dose of pirfenidone or a specific pirfenidone analog orally qd, optionally in two or more divided doses per day, for the desired treatment duration.

In one embodiment, the invention provides a method using an effective amount of insulin and pirfenidone or a specific pirfenidone analog in the treatment of Type II diabetes in a patient, comprising administering to the patient a dosage of insulin containing an amount of from about 0.5 Unit to about 50 Units per dose of insulin glargine, intramuscularly or subcutaneously qd, qod, tiw, or biw, or per day substantially continuously or continuously, in combination with a dosage of pirfenidone or a specific pirfenidone analog containing an amount of about 100 mg to about 1,000 mg, or from about 500 mg to about 5,000 of drug per dose of pirfenidone or a specific pirfenidone analog orally qd, optionally in two or more divided doses per day, for the desired treatment duration.

In one embodiment, the invention provides a method using an effective amount of insulin and pirfenidone or a specific pirfenidone analog in the treatment of Type II diabetes in a patient, comprising administering to the patient a dosage of insulin containing an amount of from about 0.5 Unit to about 50 Units per dose of Ultralente, intramuscularly or subcutaneously qd, qod, tiw, or biw, or per day substantially continuously or continuously, in combination with a dosage of pirfenidone or a specific pirfenidone analog containing an amount of about 100 mg to about 1,000 mg, or from about 500 mg to about 5,000 of drug per dose of pirfenidone or a specific pirfenidone analog orally qd, optionally in two or more divided doses per day, for the desired treatment duration.

In one embodiment, the invention provides a method using an effective amount of insulin and pirfenidone or a specific pirfenidone analog in the treatment of Type II diabetes in a patient, comprising administering to the patient a dosage of insulin containing an amount of from about 0.5 Unit to about 50 Units per dose of Lente, intramuscularly or subcutaneously qd, qod, tiw, or biw, or per day substantially continuously or continuously, in combination with a dosage of pirfenidone or a specific pirfenidone analog containing an amount of about 100 mg to about 1,000 mg, or from about 500 mg to about 5,000 of drug per dose of pirfenidone or a specific pirfenidone analog orally qd, optionally in two or more divided doses per day, for the desired treatment duration.

### Subjects Suitable for Treatment

Subjects suitable for treatment with a subject method for treating a TNF-α-mediated disorder include individuals who have been diagnosed as having, or are at risk of developing, a TNF-α-mediated disorder. TNF-α-mediated disorders are described above. Of particular interest in many embodiments is the treatment of humans.

Subjects suitable for treatment with a subject method for treating NASH include any individual who has been diagnosed as having NASH. Of particular interest in many embodiments is the treatment of humans.

Subjects suitable for treatment with a subject method for treating Type II diabetes include individuals who have been diagnosed as having Type II diabetes; individuals who have been diagnosed as having Type II diabetes, and who have been previously treated for Type II diabetes with a therapeutic agent for treating Type II diabetes, but who are refractory to treatment with, or who no longer respond to treatment with, the therapeutic agent. In some embodiments, the individual is insulin resistant. In other embodiments, the individual is insulin deficient. In other embodiments, the individual is both insulin resistant and insulin deficient. In some embodiments, the individual has a fasting glucose level that is outside of the normal range.

Individuals who are insulin resistant are identified by one or more of the following criteria: 1) a HOMA-IR value that is greater than 2.5 (based on the calculation fasting insulin (mU/ml)×fasting glucose (mmol/l)/22.5); 2) a fasting serum insulin level of greater than about 20 μU/mL, or greater than about 25 μU/mL; 3) a fasting serum C-peptide level of greater than about 3.5 ng/nL, or greater than about 4.5 ng/mL. The HOMA-IR method is described in the literature; see, e.g., Matthews et al. (1985) Diabetologia 28:412-419. In some embodiments, insulin resistance is calculated using the HOMA-IR formula: [fasting insulin (mU/ml)×fasting glucose (mmol/l)]/22.5.

### EXAMPLES

The following examples are put forth so as to provide those of ordinary skill in the art with a complete disclosure and description of how to make and use the present invention, and are not intended to limit the scope of what the inventors regard as their invention nor are they intended to represent that the experiments below are all or the only experiments performed. Efforts have been made to ensure accuracy with respect to numbers used (e.g. amounts, temperature, etc.) but some experimental errors and deviations should be accounted for. Unless indicated otherwise, parts are parts by weight, molecular weight is weight average molecular weight, temperature is in degrees Centigrade, and pressure is at or near atmospheric. Standard abbreviations may be used, e.g., bp, base pair(s); kb, kilobase(s); pl, picoliter(s); s, second(s); min, minute(s); hr, hour(s); and the like.

### Example 1: Pirfenidone in the Treatment of NASH

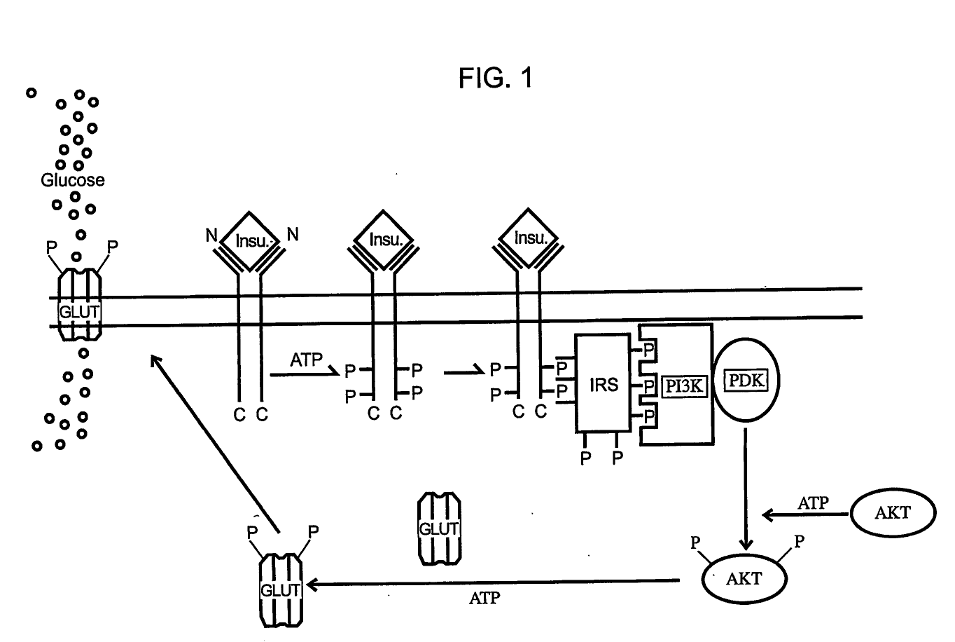

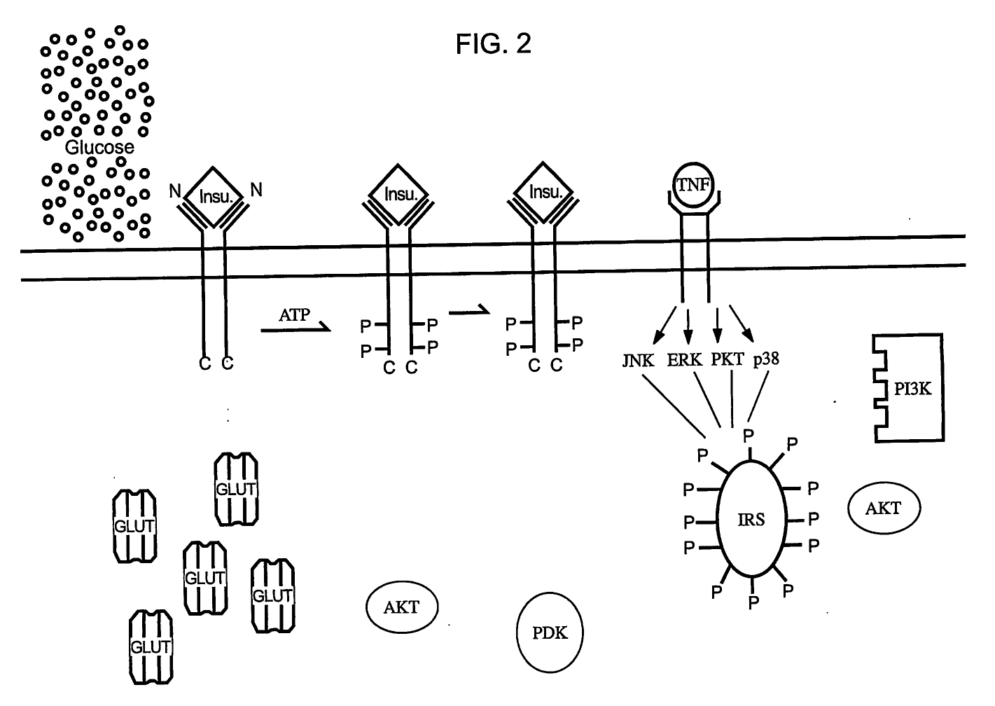

Nonalcoholic Steatohepatitis (NASH) is described as the presence of large droplet steatosis accompanied by evidence of hepatocellular necrosis and fibrosis. Several studies confirmed that NASH can progress to hepatic insufficiency, cirrhosis and hepatocellular carcinoma. Over the last decade, there is increasing evidence that insulin resistance may play an important role in the pathogenesis of NASH. Individuals at the greatest risk for developing NASH have conditions related to diabetes and obesity. Tumor Necrosis Factor-α (TNF-α) is the first identified fat-derived peptide, and accumulating evidences suggests that fat-derived TNF-α is directly involved in the development of insulin resistance in obesity and NASH. It has been postulated that TNF-α-mediated serine-threonine phosphorylation of Insulin Receptor Substrates (IRS) contributes significantly to TNF-induced diabetes. FIG. 1 depicts insulin signaling in the absence of TNF. FIG. 2 depicts insulin signaling in the presence of high concentrations of TNF.

Pirfenidone (5-methyl-1phenyl-2(1H)-pyridone), is a small molecule (MW 185) that has demonstrated antifibrotic activity both in vitro and in human clinical trials. Pirfenidone has been shown to down regulate the production of TNF-α as well as to regulate the production of TGF-β. To explore the possibility that Pirfenidone could be used to treat NASH, the effects of pirfenidone on cells stimulated with Insulin in the presence or absence of TNF-α were examined.

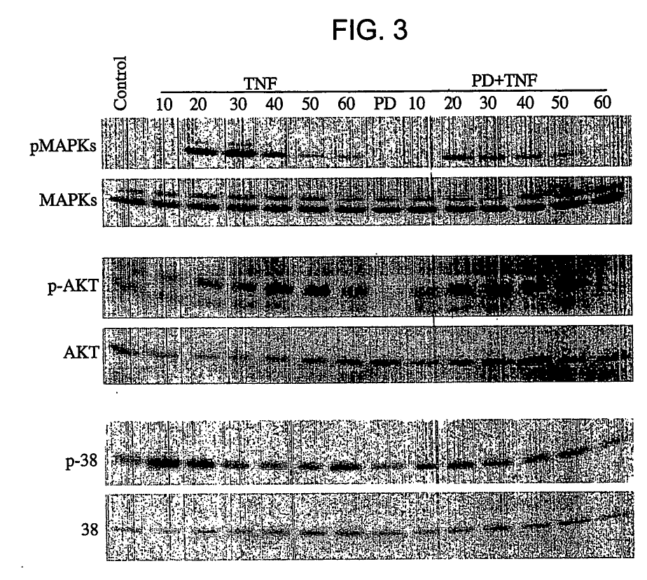

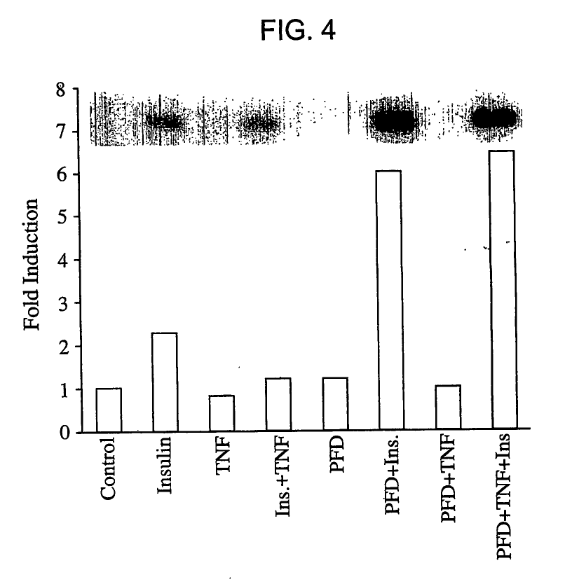

These experiments indicated that treatment of cells with TNF-α induces Insulin resistance by hyper-phosphorylation of the IRS. When cells were treated with Pirfenidone, TNF-α mediated Insulin resistance was ameliorated. Western Blot studies demonstrated that the mechanism of Pirfenidone's action was to block the activity of several kinases in the TNF cascade including activation of serine-threonine kinases. More specifically, Pirfenidone decreased ERK activation by 7-fold, p38 MAPK by 2-fold and RAF activation by 2-fold (P<0.01 for all observations). In addition, Pirfenidone decreased the TNF-α mediated phosphorylation of the transcription activation factor CREB by 2-fold (P<0.01). These studies demonstrate that Pirfenidone blocks TNF-α-mediated inhibition of insulin-induced IRS1 tyrosine phosphorylation. In addition, Pirfenidone increased insulin-induced phosphorylation of IRS1. These data suggest that pirfenidone can be an effective treatment to reverse steatosis in NASH patients. The results are depicted in FIGS. 3 and 4. FIG. 3 depicts inhibition of MAPK by pirfenidone. FIG. 4 depicts enhancement of insulin signaling by pirfenidone.

### Example 2: Effect of Pirfenidone on the JNK (c-jun) Kinase Pathway

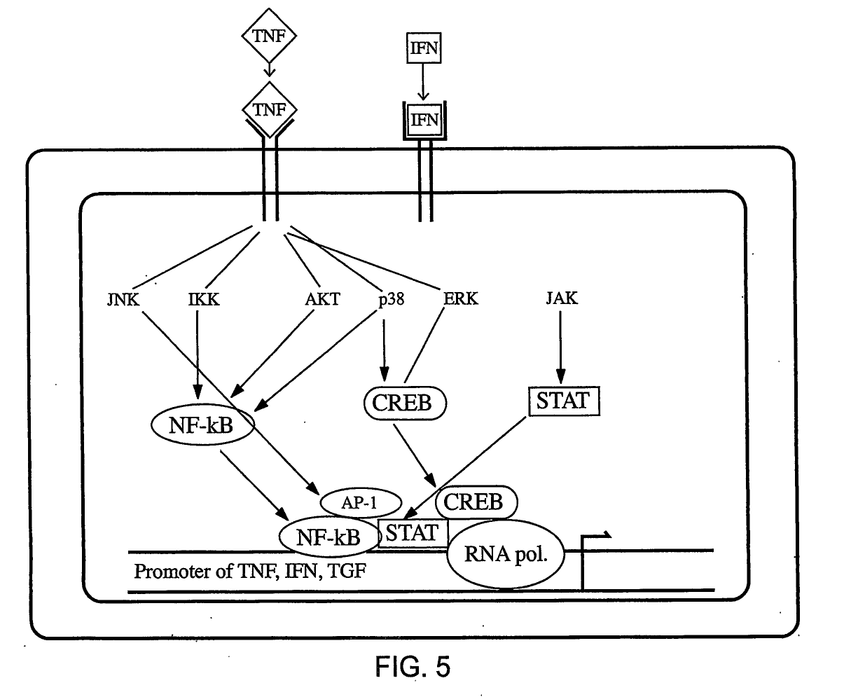

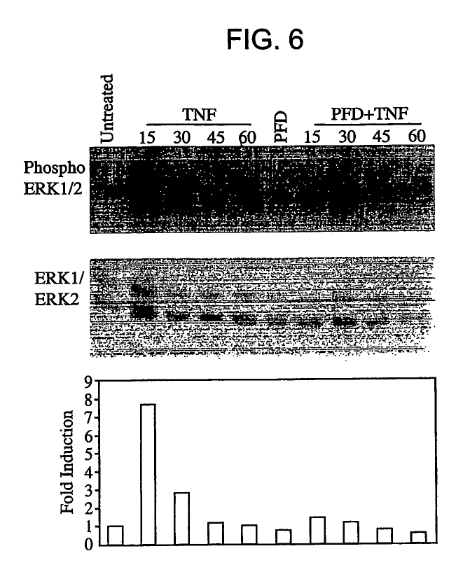

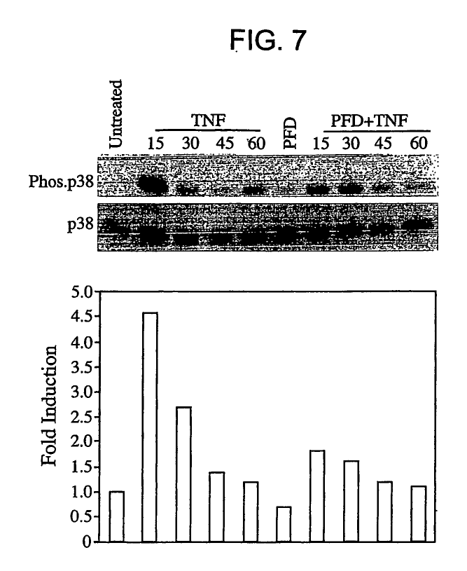

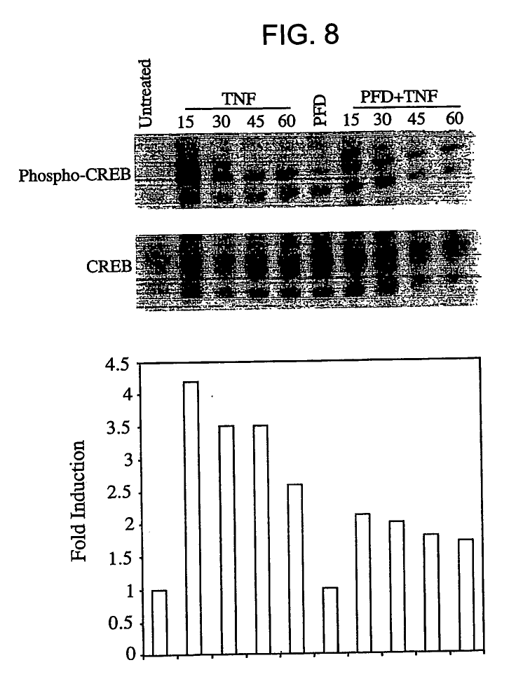

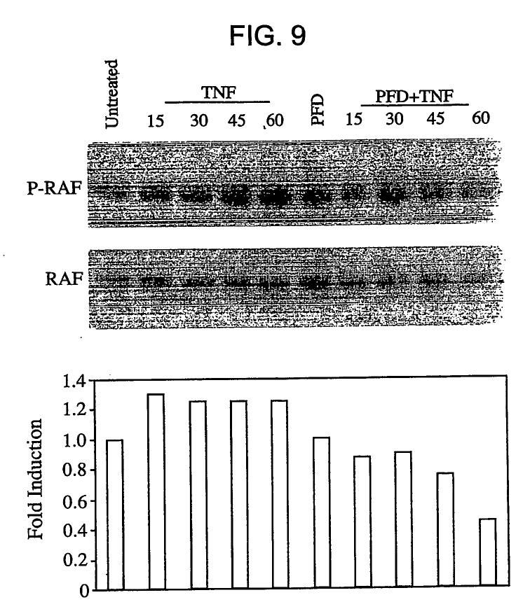

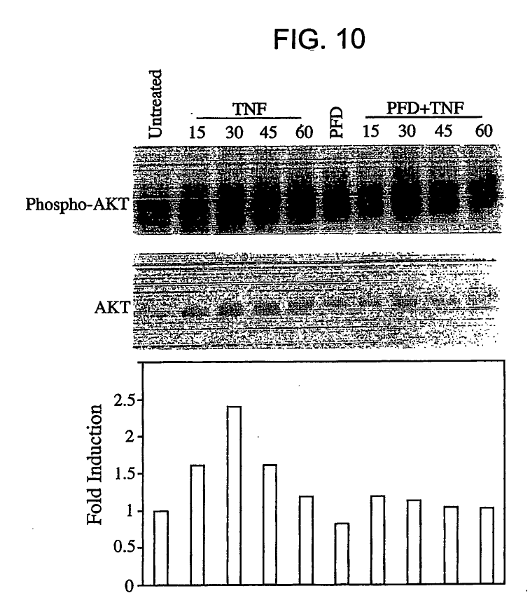

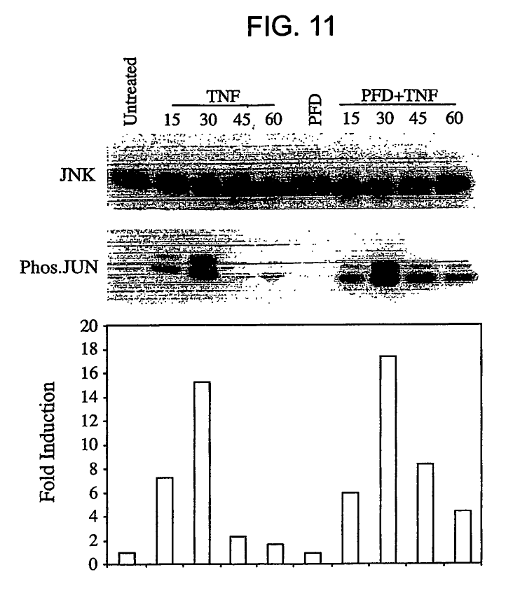

FIG. 5 depicts various downstream signaling events that are triggered by TNF binding to a TNF receptor. TNF-mediated activation of various serine-threonine kinases are differentially affected by pirfenidone. Therefore, activation of transcription factors downstream of a given kinase will be differentially affected by pirfenidone. FIG. 6 depicts pirfenidone inhibition of TNF-induced ERK activation. FIG. 7 depicts pirfenidone inhibition of TNF-induced p38 MAPK activation. FIG. 8 depicts pirfenidone inhibition of TNF-induced activation of the transcription factor CREB. FIG. 9 depicts pirfenidone inhibition of TNF-induced activation of RAF kinase. FIG. 10 depicts pirfenidone inhibition of TNF-induced activation of AKT. FIG. 11 depicts pirfenidone potentiation of TNF-induced JNK activation.

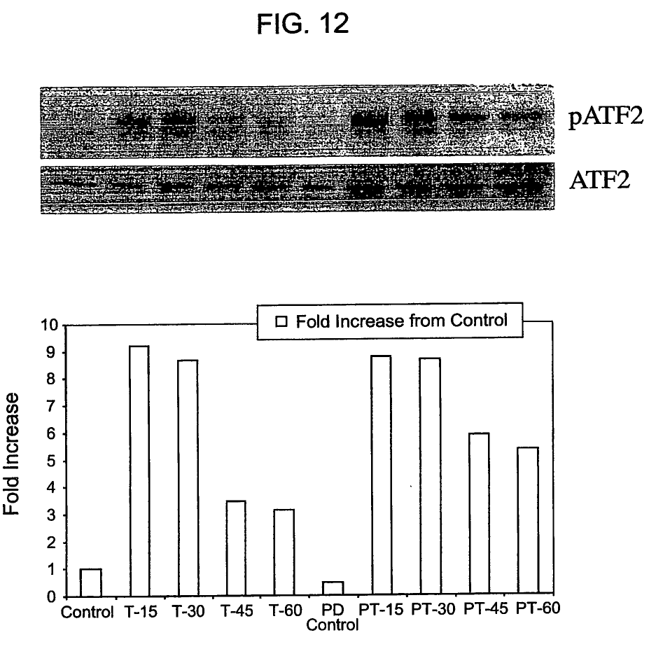

While phosphorylation of CREB is diminished due to decreased activity of p38 in the presence of pirfenidone, phosphorylation of the JNK-phosphorylated transcription factor ATF2 is potentiated in the presence of pirfenidone. The results are shown in FIG. 12. FIG. 12 depicts pirfenidone potentiation of ATF2. Thus, pirfenidone potentiates TNF-induced activation of the JNK kinase pathway.

While the present invention has been described with reference to the specific embodiments thereof, it should be understood by those skilled in the art that various changes may be made and equivalents may be substituted without departing from the true spirit and scope of the invention. In addition, many modifications may be made to adapt a particular situation, material, composition of matter, process, process step or steps, to the objective, spirit and scope of the present invention. All such modifications are intended to be within the scope of the claims appended hereto.

1. A method of treating a TNF-mediated disorder in an individual, the method comprising administering to an individual in need thereof a sub-therapeutic amount of a TNF-α antagonist that is lower than a minimum dose effective, free of concomitant therapy with pirfenidone or a pirfenidone analog, to reduce one or more signs or symptoms of the disorder in the individual; and
co-administering an amount of pirfenidone or a pirfenidone analog that in combination with the amount of the TNF-α antagonist is effective to reduce one or more signs or symptoms of the TNF-mediated disorder in the individual, while reducing or avoiding the severity or incidence of infection that would arise from the use of the TNF-α antagonist at or above the minimum dose.

2. (canceled)

3. The method of claim 1, wherein the TNF-α antagonist is a monoclonal antibody that binds TNF-α.

4. The method of claim 3, wherein the monoclonal antibody is REMICADE®.

5. The method of claim 3, wherein the monoclonal antibody is HUMIRA™.

6. The method of claim 1, wherein the TNF-α antagonist is a soluble TNF receptor.

7. The method of claim 6, wherein the soluble TNF receptor is ENBREL®.

8. The method of claim 1, wherein the TNF-mediated disorder is rheumatoid arthritis.

9. The method of claim 1, wherein the TNF-mediated disorder is Crohn's disease.

10. A method of treating Type II diabetes in an individual, the method comprising administering to an individual having Type II diabetes an effective amount of pirfenidone or a pirfenidone analog and an effective amount of insulin, wherein the individual is insulin resistant and wherein the individual is insulin deficient.

11. The method of claim 10, wherein the insulin is selected from insulin aspart, insulin detemir, insulin lispro, insulin glargine, Ultralente, isophane insulin, Humulin 50/50, and Humulin 70/30.

12. A method of treating non-alcoholic steatohepatitis, the method comprising administering to an individual in need thereof an effective amount of pirfenidone or a pirfenidone analog.
